(19) United States
(12) Patent Application Publication
(10) Pub. No.: US 2024/0010579 A1
(43) Pub. Date: Jan. 11, 2024

(54) Processes for Making Linear Alpha-Olefins

(71) Applicant: ExxonMobil Chemical Patents Inc., Baytown, TX (US)

(72) Inventors: Jeffrey C. Bunquin, Houston, TX (US); Joshua J. Willis, Menlo Park, CA (US); Partha Nandi, Annandale, NJ (US); Javier Guzman, The Woodlands, TX (US)

(21) Appl. No.: 18/042,386

(22) PCT Filed: Aug. 10, 2021
(86) PCT No.: PCT/US2021/045322

§ 371 Date: Feb. 21, 2023

Related U.S. Application Data
(60) Provisional application No. 63/079,652, filed on Sep. 17, 2020.

(51) Int. Cl. C07C 1/04; B01J 23/83; B01J 23/889; B01J 35/00; B01J 37/18; B01J 37/08; B01J 37/02

(52) U.S. Cl. CPC C07C 1/044; B01J 23/83; B01J 23/8892; B01J 35/0013; B01J 35/006; B01J 37/18; B01J 37/082; B01J 37/0205; B01J 37/0236; C07C 2523/06; C07C 2523/28; C07C 2523/30; C07C 2523/34; C07C 2523/04; C07C 2523/72; C07C 2523/745; C07C 2523/75; C07C 2523/755; C07C 2523/50; C07C 2523/10

(57) ABSTRACT

Fischer-Tropsch processes for converting syngas produces linear alpha olefins at high yield and selectivity in the presence of supported nano-particle catalyst compositions and/or metal carbide/nitride-containing catalyst compositions.

### CROSS-REFERENCE TO RELATED APPLICATION

This application claims priority to and the benefit of U.S. Provisional Application No. 63/079,652 having a filing date of Sep. 17, 2020, the disclosure of which is incorporated herein by reference in its entirety.

### FIELD

This disclosure relates to processes for making linear alpha olefins. In particular, this disclosure relates to processes for making linear alpha olefins from syngas in the presence of a catalyst composition.

BACKGROUND

Linear alpha-olefins (“LAOs”) are linear olefins comprising a terminal carbon-carbon double bond. LAOs have found use in making synthetic lubricating oil base stocks, drilling fluid base stock, and the like, with various viscosity grades by oligomerization thereof to various degrees in the presence of an oligomerization catalyst such as BF₃, AlCl₃, and metallocene catalyst systems. Such synthetic base stocks are sometimes called polyalpha-olefins (“PAOs”). LAOs can also be used for making alkylated aromatic hydrocarbons, which are useful for making surfactants. Additionally, LAOs such as 1-butene have been used as a co-monomer with ethylene in making polyethylene at various grades, such as high-density polyethylene, and linear low-density polyethylene, and the like. A significant use of C4-C8 LAOs is in producing linear aldehydes trough, e.g., hydroformylation, which can be subsequently converted into short-chain fatty acids, or linear alcohols for plasticizer applications by aldehyde hydrogenation.

One approach for producing LAOs involves ethylene oligomerization followed by separation. Another approach is the Fischer-Tropsch process, in which syngas is converted into liquid hydrocarbons and/or oxygenates in the presence of a Fischer-Tropsch catalyst. Traditional Fischer-Tropsch catalysts comprise Co, Fe, and Re.

Synthesis gas (syngas) is a mixture of hydrogen and carbon monoxide generated from the upgrading of chemical feedstocks such as natural gas and coal. Syngas has been used industrially for the production of value-added chemicals including chemical intermediates, such as olefins, alcohols, and fuels. Fischer-Tropsch catalysis is one route for syngas conversion to value-added products. Generally, Fischer-Tropsch catalysis involves the use of iron and cobalt catalysts for the production of gasoline range products for transportation fuels, heavy organic products including distillates used in diesel fuels, and high purity wax for a range of applications including food production. Similar catalysts can be used for the production of value-added chemical intermediates including olefins and alcohols that can be used, for example, for the production of polymers and fuels. Often the production of value-added chemicals includes the production of saturated hydrocarbons, such as paraffins. The selectivity of Fischer-Tropsch catalysts towards production of value-added chemical intermediates may be adjusted by addition of promoters comprising group 1 and group 2 cations and transition metals. Fischer-Tropsch catalysts have been prepared as metal oxides or sulfides of iron and cobalt. The iron and cobalt catalysts are frequently supported on solid carriers comprising oxides such as alumina, silica, or various clays or on carbonaceous materials. Fischer-Tropsch catalysts have been used to produce hydrocarbons in the gasoline range and lighter hydrocarbons.

There remains a need for improved Fischer-Tropsch catalyst compositions and processes for making LAOs. This disclosure satisfies this and other needs.

### SUMMARY

We have found that size-, shape-, and/or composition-controlled nanoparticles can be fabricated and subsequently dispersed onto support materials to make Fischer-Tropsch catalyst compositions useful for making LAOs. We have also found that metal carbide(s)- and/or metal nitride(s)-containing catalyst compositions made by decomposing a catalyst precursor can also be used for making LAOs in a Fischer-Tropsch process.

A first aspect of this disclosure relates to process for making linear alpha-olefin(s), the process comprising:

(I) contacting a feed comprising syngas with a catalyst composition in a conversion reactor under conversion conditions to produce a conversion product mixture comprising a linear alpha-olefin, wherein the catalyst composition comprises a catalytic component, wherein the catalytic component comprises:

a metal element M¹, selected from iron, cobalt, manganese, and combinations of two or more thereof at any proportion;

a metal element M², selected from aluminum, gallium, indium, thallium, chromium, scandium, yttrium, the lanthanide series, the actinide series, and any combination of two or more thereof at any proportion;

an optional metal M³, differing from M¹ and M²;

carbon;

nitrogen; and

optionally sulfur, at a molar ratio of M², M³, carbon, nitrogen, and sulfur to M¹ of r1, r2, r3, r4, and r5, respectively, indicated below:

M²:M³ :C:N:S:M¹=r1:r2:r3:r4:r5:1, where:

0.1≤r1≤1.5;

0≤r2≤0.5;

0<r3≤1;

0<r4≤1; and

0≤r5≤1; and

(II) obtaining a linear alpha-olefin product from the conversion product mixture.

A second aspect of this disclosure relates to a process for making linear alpha-olefin(s), the process comprising:

(I) contacting a feed comprising syngas with a catalyst composition in a conversion reactor under conversion conditions to produce a conversion product mixture comprising a linear alpha-olefin, wherein the catalyst composition comprises:

a support; and

a plurality of nanoparticles on the support, wherein:

each nanoparticle comprises a kernel, the kernels have an average particle size from 4 to 100 nm and a particle size distribution of no greater than 20%; the kernels comprise oxygen, a metal element M1, optionally sulfur, optionally phosphorus, an optional metal element M2, and optionally a third metal element M3, where:
M1 is selected from Mn, Fe, Co, and combination of two or more thereof;
M2 is selected from Ni, Zn, Cu, Mo, W, Ag, and combinations thereof;
M3 is selected from Y, Sc, alkaline metals, the lanthanides, group 13, 14, or 15 elements, and combinations thereof; and
the molar ratios of M2, M3, S, and P, if any, to M1 is r1, r2, r3, and r4, respectively, 0≤r1≤2, 0≤r2≤2, 0≤r3≤5, and 0≤14≤5; and

(II) obtaining a linear alpha-olefin product from the conversion product mixture

### BRIEF DESCRIPTION OF THE DRAWINGS

FIG. 1 is a graph showing X-ray diffraction (“XRD”) patterns of 7 bimetallic, iron-containing catalyst precursors of this disclosure.

FIG. 2 is a graph showing XRD patterns of 3 trimetallic, cobalt-containing catalyst precursors of this disclosure.

FIG. 3 is a graph showing XRD patterns of 6 used catalytic components of this disclosure.

FIG. 4 is a graph showing the thermogravimetric analysis results of an iron-containing catalyst precursor of this disclosure.

FIG. 5 is a graph showing thermogravimetric analysis result of a cobalt-containing catalyst precursor of this disclosure.

FIG. 6 is a graph showing an XRD pattern of a cobalt-containing catalytic component of this disclosure, and three peak groups identified therein corresponding to three different phases.

FIG. 7 is a graph showing the XRD pattern of the catalytic component shown in FIG. 7, and seven additional peak groups identified therein corresponding to seven additional phases.

FIG. 8 is a graph showing the XRD pattern of the catalytic component shown in FIGS. 6 and 7, and three additional peak groups identified therein corresponding to three additional phases.

### DETAILED DESCRIPTION

In this disclosure, a process is described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other step, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described.

Unless otherwise indicated, all numbers indicating quantities in this disclosure are to be understood as being modified by the term “about” in all instances. It should also be understood that the numerical values used in the specification and claims constitute specific embodiments. Efforts have been made to ensure the accuracy of the data in the examples. However, it should be understood that any measured data inherently contain a certain level of error due to the limitation of the technique and equipment used for making the measurement.

As used herein, the indefinite article “a” or “an” shall mean “at least one” unless specified to the contrary or the context clearly indicates otherwise. Thus, embodiments comprising “a metal” include embodiments comprising one, two, or more metals, unless specified to the contrary or the context clearly indicates only one metal is included.

For the purposes of this disclosure, the nomenclature of elements is pursuant to the version of Periodic Table of Elements as described in CHEMICAL AND ENGINEERING NEWS, 63(5), pg. 27 (1985). Abbreviations for atoms are as given in the periodic table (Li=lithium, for example).

The following abbreviations may be used herein for the sake of brevity: RT is room temperature (and is 23° C. unless otherwise indicated), kPag is kilopascal gauge, psig is pound-force per square inch gauge, psia is pound-force per square inch absolute, and WHSV is weight hourly space velocity, and GHSV is gas hourly space velocity. Abbreviations for atoms are as given in the periodic table (Co=cobalt, for example).

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of this disclosure. Additionally, they do not exclude impurities and variances normally associated with the elements and materials used. “Consisting essentially of” a component in this disclosure can mean, e.g., comprising, by weight, at least 80 wt %, of the given material, based on the total weight of the composition comprising the component.

For purposes of this disclosure and claims thereto, the term “substituted” means that a hydrogen atom in the compound or group in question has been replaced with a group or atom other than hydrogen. The replacing group or atom is called a substituent. Substituents can be, e.g., a substituted or unsubstituted hydrocarbyl group, a heteroatom, a heteroatom-containing group, and the like. For example, a “substituted hydrocarbyl” is a group derived from a hydrocarbyl group made of carbon and hydrogen by substituting at least one hydrogen in the hydrocarbyl group with a non-hydrogen atom or group. A heteroatom can be nitrogen, sulfur, oxygen, halogen, etc.

The terms “hydrocarbyl,” “hydrocarbyl group,” or “hydrocarbyl radical” interchangeably mean a group consisting of carbon and hydrogen atoms. For purposes of this disclosure, “hydrocarbyl radical” is defined to be C1-C100 radicals, that may be linear, branched, or cyclic, and when cyclic, aromatic or non-aromatic.

The term “melting point” (mp) refers to the temperature at which solid and liquid forms of a substance can exist in equilibrium at 760 mmHg.

The term “boiling point” (bp) refers to the temperature at which liquid and gas forms of a substance can exist in equilibrium at 760 mmHg.

“Soluble” means, with respect to a given solute in a given solvent at a given temperature, at most 100 mass parts of the solvent is required to dissolve 1 mass part of the solute under a pressure of 1 atmosphere. “Insoluble” means, with respect to a given solute in a given solvent at a given temperature, more than 100 mass parts of the solvent is required to dissolve 1 mass part of the solute under a pressure of 1 atmosphere.

The term “branched hydrocarbon” means a hydrocarbon comprising at least 4 carbon atoms and at least one carbon atom connecting to three carbon atoms.

The term “olefinicity” refers to the molar ratio of the sum of olefins to the sum of paraffins detected. The olefinicity increases when the olefin/paraffin molar ratio increases. The olefinicity decreases when the olefin/paraffin molar ratio decreases.

The terms “alkyl,” “alkyl group,” and “alkyl radical” interchangeably mean a saturated monovalent hydrocarbyl group. A “cyclic alkyl” is an alkyl comprising at least one cyclic carbon chain. An “acyclic alkyl’ is an alkyl free of any cyclic carbon chain therein. A “linear alkyl” is an acyclic alkyl having a single unsubstituted straight carbon chain. A “branched alkyl” is an acyclic alkyl comprising at least two carbon chains and at least one carbon atom connecting to three carbon atoms. Alkyl groups can comprise, for example, methyl, ethyl, n-propyl, isopropyl, n-butyl, isobutyl, sec-butyl, tert-butyl, pentyl, iso-amyl, hexyl, octyl, cyclopropyl, cyclobutyl, cyclopentyl, cyclohexyl, cyclooctyl, and the like including their substituted analogues.

The term “Cn” compound or group, where n is a positive integer, means a compound or a group comprising carbon atoms therein at the number of n. Thus, a “Cm to Cn” alkyl means an alkyl group comprising carbon atoms therein at a number in a range from m to n, or a mixture of such alkyl groups. Thus, a C1-C3 alkyl means methyl, ethyl, n-propyl, or 1-methylethyl-. The term “Cn+” compound or group, where n is a positive integer, means a compound or a group comprising carbon atoms therein at the number of equal to or greater than n. The term “Cn−” compound or group, where n is a positive integer, means a compound or a group comprising carbon atoms therein at the number of equal to or lower than n.

The term “conversion” refers to the degree to which a given reactant in a particular reaction (e.g., dehydrogenation, hydrogenation, etc.) is converted to products. Thus 100% conversion of carbon monoxide means complete consumption of carbon monoxide, and 0% conversion of carbon monoxide means no measurable reaction of carbon monoxide.

The term “selectivity” refers to the degree to which a particular reaction forms a specific product, rather than another product. For example, for the conversion of syngas, 50% selectivity for C1-C4 alcohols means that 50% of the products formed are C1-C4 alcohols, and 100% selectivity for C1-C4 alcohols means that 100% of the products formed are C1-C4 alcohols. The selectivity is based on the product formed, regardless of the conversion of the particular reaction.

The term “nanoparticle” means a particle having a largest dimension in the range from 0.1 to 500 nanometers.

The term “long-chain” means comprising a straight carbon chain having at least 8 carbon atoms excluding any carbon atoms in any branch that may be connected to the straight carbon chain. Thus, n-octane and 2-octain are long-chain alkanes, but 2-methylheptane is not. A long-chain organic acid is an organic acid comprising a straight carbon chain having at least 8 carbon atoms excluding any carbon atoms in any branch that may be connected to the straight carbon chain. Thus, octanoic acid is a long-chain organic acid, but 6-methylheptanoic acid is not.

The term “organic acid” means an organic Bronsted acid capable of donating a proton. Organic acids include, carboxylic acids of any suitable chain length; carbon containing sulfinic, sulfonic, phosphinic, and phosphonic acids; hydroxamic acids, and in some embodiments, amidines, amides, imides, alcohols, and thiols.

The term “surfactant” means a material capable of reducing the surface tension of a liquid in which it is dissolved. Surfactants can find use in, for example, detergents, emulsifiers, foaming agents, and dispersants. The term “linear alpha-olefin” or “LAO” interchangeably means a linear olefin

comprising a terminal C═C bond. The term “linear internal olefin” or “LIO” interchangeably means a linear olefin free of a terminal C═C bond. A LAO product can comprise, consists essentially of, or consist of one or more LAO. For example, a LAO product can comprise, consist essentially of, or consist of, 1-butene, 1-pentene, 1-hexene, 1-octene, or a mixture thereof.

Detailed description of the nanoparticles and catalyst compositions of this disclosure, including the composition comprising nanoparticles of the first aspect, the process for producing nanoparticles of the second aspect, and the catalyst composition of the third aspect of this disclosure, is provided below.

### I. The First Aspect of this Disclosure

The first aspect of this disclosure generally relates to a process for making LAOs from syngas comprising one or more of the following steps:

(I) contacting a feed comprising syngas with a catalyst composition in a conversion reactor under conversion conditions to produce a conversion product mixture comprising a linear alpha-olefin, wherein the catalyst composition comprises a catalytic component, wherein the catalytic component comprises:

a metal element M¹, selected from iron, cobalt, manganese, and combinations of two or more thereof at any proportion;

a metal element M², selected from aluminum, gallium, indium, thallium, chromium, scandium, yttrium, the lanthanide series, the actinide series, and any combination of two or more thereof at any proportion;

an optional metal M³, differing from M¹ and M^{2;}

carbon;

nitrogen; and

optionally sulfur, at a molar ratio of M², M³, carbon, nitrogen, and sulfur to M¹ of r1, r2, r3, r4, and r5, respectively, indicated below:

M²:M³:C:N:S:M¹=r1:r2:r3:r4:r5:1, where:

0.1≤r1≤1.5;

0≤r2≤0.5;

0<r3≤1;

0<r4≤1; and

0≤r5≤1; and

(II) obtaining a linear alpha-olefin product from the conversion product mixture.

More details of the process of the first aspect of this disclosure is given below.

### I.1 Catalyst Compositions

In the catalyst compositions of this disclosure, preferably M¹ is selected from iron, cobalt, combinations of iron and cobalt at any proportion, combinations of iron and manganese at any proportion, combination of cobalt with manganese at any proportion, and combination of iron, cobalt, and manganese at any proportion. In specific embodiments, M¹ is a single metal of cobalt or iron. Where M¹ comprises a binary mixture/combination of cobalt and manganese, preferably cobalt is present at a higher molar proportion than manganese. Where M¹ comprises a binary mixture/combination of iron and manganese, preferably iron is present at a higher molar proportion than manganese. Without intending to be bound by a particular theory, it is believed that the presence of M¹ provides at least a portion of the catalytic effect of the catalytic component of the catalyst composition of the first aspect of this disclosure.

Preferably M² is selected from aluminum, gallium, indium, thallium, chromium, scandium, yttrium, and the lanthanide series. More preferably M² is selected from gallium, indium, scandium, yttrium, and the lanthanide series. Particularly desirable lanthanide series for the catalyst composition of the first aspect of this disclosure include, but are not limited to: La, Ce, Pr, Nd, Gb, Dy, Ho, and Er. Without intending to be bound by a particular theory, it is believed that the presence of M² promotes the catalytic effect of M¹ in the catalyst compositions of this disclosure.

The presence of M³ in the catalyst compositions of this disclosure is optional. If present, M³ is preferably selected from alkali metals, copper, silver, and any combinations and mixtures of two or more thereof at any proportion. In certain embodiments, M³ is selected from copper, silver, and mixtures/combinations thereof. Without intending to be bound by a particular theory, it is believed the presence of metal M³ can promote the catalyst effect of the catalyst compositions of this disclosure.

Metal carbides such as iron carbide and cobalt carbide have been reported as catalysts for converting syngas to make various organic compounds. The catalyst compositions of this disclosure comprise a catalytic component comprising carbon. It is believed that in the catalytic component of a catalyst composition of this disclosure, carbon may be present at least in part as a carbide of a metal. The presence of a metal carbide can be indicated by the XRD graph of the catalyst composition. By a “metal carbide,” it is meant to include carbide of a single metal, or a combination of two or more metals M¹, M², and/or M³. Desirably the catalytic component comprises a carbide of a single metal, or a combination of two or more metals of M¹, and/or M². Desirably the catalytic component comprises a carbide of a single metal, or a combination of two or more metals of M¹. Desirably the catalytic component comprises one or more of iron carbide, cobalt carbide, manganese carbide, (mixed iron cobalt) carbide, (mixed iron manganese) carbide, mixed (cobalt manganese) carbide, and mixed (cobalt, iron, and manganese) carbide. Desirably, the catalytic component comprises a carbide of a single metal, or a combination of two or more metals of M² (e.g., yttrium and the lanthanides). The catalytic component may comprise a carbide of a metal mixture comprising an M¹ and an M². The identification of the presence of a carbide phase in a catalyst composition can be conducted by comparing the XRD data of the catalyst composition against an XRD peak database of known carbides, such as those available from International Center for Diffraction Data (“ICDD”).

A novel feature of the catalyst compositions for converting syngas of the first aspect of this disclosure resides in the presence of nitrogen in the catalytic component of the of the catalyst composition, in addition to carbon. It is believed that in the catalytic component of the catalyst composition of this disclosure, nitrogen may be present in part as a nitride of a metal. The presence of a metal nitride can be indicated by the XRD graph of the catalyst composition. By a “metal nitride,” it is meant to include nitride of a single metal, or a combination of two or more metals of M¹, M², and M³. Desirably the catalytic component comprises a nitride of a single metal, or a combination of two or more metals of M¹, and/or M². Desirably the catalytic component comprises a nitride of a single metal, or a combination of two or more metals of M¹. Desirably the catalyst comprises one or more of iron nitride, cobalt nitride, manganese nitride, (mixed iron cobalt) nitride, (mixed iron manganese) nitride, mixed (cobalt manganese) nitride, and mixed (cobalt, iron, and manganese) nitride. Desirably, the catalytic component comprises a nitride of a single metal, or a combination of two or more metals of M² (e.g., yttrium and the lanthanides). The catalytic component may comprise a nitride of a metal mixture comprising an M¹ and an M². The identification of the presence of a nitride phase in a catalyst composition can be conducted by comparing the XRD data of the catalyst composition against an XRD peak database of known nitrides.

The catalyst compositions of this disclosure may optionally comprise sulfur in the catalytic component thereof. Without intending to be bound by a particular theory, in certain embodiments, the presence of sulfur can promote the catalytic effect of the catalyst composition. The sulfur may be present as a sulfide of one or more metals of M¹, M², and/or M³.

In specific embodiments, the catalytic component of a catalyst composition of this disclosure consists essentially of M¹, M², M³, carbon, nitrogen, and optionally sulfur, e.g., comprising ≥85, or ≥90, or ≥95, or ≥98, or even ≥99 wt % of M¹, M², M³, carbon, nitrogen, and optionally sulfur, based on the total weight of the catalytic component.

The molar ratios of M², M³, carbon, nitrogen, and sulfur to M¹, r1, r2, r3, r4, and r5, respectively, in the catalytic component of a catalyst composition of this disclosure are calculated from the aggregate molar amounts of the elements in question. Thus, if M¹ is a combination/mixture of two or more metals, the aggregate molar amounts of all metals M¹ is used for calculating the ratios. If M² is a combination/mixture of two or more metals, the aggregate molar amounts of all metals M² is used for calculating the ratio rl. If M³ is a combination/mixture of two or more metals, the aggregate molar amounts of all metals M³ is used for calculating the ratio r2.

Preferably, the molar ratio of M² to M¹ in the catalytic component of a catalyst compositions of this disclosure, r1, can range from r1a to r1b, where r1a and r1b can be, independently, e.g., 0.1, 0.2, 0.3, 0.4, 0.5, 0.6, 0.7, 0.8, 0.9, 1.0, 1.1, 1.2, 1.3, 1.4 or 1.5, as long r1a<r1b. More preferably r1a=0.8, r1b=1.2; still more preferably r1a=0.9, r1b=1.1. In one particularly advantageous embodiment, r1 is in the vicinity of 1.0 (e.g., from 0.95 to 1.05), meaning that M¹ and M² are present in the catalytic component at substantially equivalent molar amounts.

Preferably, the molar ratio of M³ to M¹ in the catalytic component of a catalyst compositions of this disclosure, r2, can range from r2a to r2b, where r2a and r2b can be, independently, e.g., 0, 0.1, 0.2, 0.3, 0.4, or 0.5, as long as r2a<r2b. Thus M³, if present, is at a substantially lower molar amount than M¹.

Preferably, the molar ratio of carbon to M¹ in the catalytic component of a catalyst composition of this disclosure, r3, can range from r3a to r3b, where r3a and r3b can be, independently, e.g., 0.1, 0.2, 0.3, 0.4, 0.5, 0.6, 0.7, 0.8, 0.9, or 1.0, as long as r3a<r3b.

Preferably, the molar ratio of nitrogen to M¹ in the catalytic component of a catalyst composition of this disclosure, r4, can range from r4a to r4b, wherein r4a and r4b can be, independently, e.g., 0.1, 0.2, 0.3, 0.4, 0.5, 0.6, 0.7, 0.8, 0.9, or 1.0, as long as r4a<r4b.

Preferably, the molar ratio of sulfur to M¹ in the catalytic component of a catalyst composition of this disclosure, r5, can range from r5a to r5b, wherein r5a and r5b can be, independently, e.g., 0, 0.1, 0.2, 0.3, 0.4, 0.5, 0.6, 0.7, 0.8, 0.9, 1.0, as long as r5a<r5b. Preferably r5a=0, and r5b=0.5. Still more preferably r5a=0 and r5b=0.3.

In specific embodiments, the metal(s) M¹ can be distributed substantially homogeneously in the catalytic component. Additionally and/or alternatively, the metal(s) M² can be distributed substantially homogeneously in the catalytic component. Additionally and/or alternatively, carbon can be distributed substantially homogeneously in the catalytic component. Still additionally and/or alternatively, nitrogen can be distributed substantially homogeneously in the catalytic component.

It is highly advantageous that the metal carbide(s) and/or the metal nitride(s) are highly dispersed in the catalytic component. The metal carbide(s) and/or the metal nitride(s) can be substantially homogeneously distributed in the catalytic component, resulting in a highly dispersed distribution, which can contribute to a high catalytic activity of the catalytic component.

Without intending to be bound by a particular theory, it is believed that the metal carbide(s), the metal nitride(s), and possibly the elemental phases of M¹ in the catalytic component provide the desired catalytic activity for chemical conversion processes such as syngas conversion processes. One or more of M² and/or M³ can provide direct catalytic function as well. In addition, one or more of M² and/or M³ can perform the function of a “promoter” in the catalytic component. Furthermore, sulfur, if present, can perform the function of a promoter in the catalytic component as well. Promoters typically improve one or more performance properties of a catalyst. Example properties of catalytic performance enhanced by inclusion of a promoter in a catalyst over the catalyst composition without a promoter, may include selectivity, activity, stability, lifetime, regenerability, reducibility, and resistance to potential poisoning by impurities such as sulfur, nitrogen, and oxygen.

The catalyst composition of this disclosure may consist essentially of the catalytic component of this disclosure, e.g., comprising ≥85, or ≥90, or ≥95, or ≥98, or even ≥99 wt % of the catalytic component, based on the total weight of the catalyst composition. Such catalyst composition may be considered as a “bulk catalyst” in that it comprises minor amount of carrier or support material in its composition, if any at all. Bulk catalysts can be conveniently made by thermal decomposition from a catalyst precursor, as described below.

The catalyst composition of this disclosure can comprise a catalyst support material (which may be called a carrier or a binder), at any suitable quantity, e.g., ≥20, ≥30, ≥40, ≥50, ≥60, ≥70, ≥80, ≥90, or even ≥95 wt %, based on the total weight of the catalyst composition. In supported catalyst compositions, the catalytic component can be desirably disposed on the internal or external surfaces of the catalyst support material. Catalyst support materials may include porous materials that provide mechanical strength and a high surface area. Non-limiting examples of suitable support materials can include oxides (e.g. silica, alumina, titania, zirconia, and mixtures thereof), treated oxides (e.g. sulphated), crystalline microporous materials (e.g. zeolites), non-crystalline microporous materials, cationic clays or anionic clays (e.g. saponite, bentonite, kaoline, sepiolite, hydrotalcite), carbonaceous materials, or combinations and mixtures thereof. Deposition of the catalytic component on a support can be effected by, e.g., incipient impregnation. A support material can be sometimes called a binder in a catalyst composition.

### I.2 Catalyst Precursors

The catalytic component of the catalyst composition, or the catalyst composition per se, of this disclosure may be produced from a catalyst precursor. The catalyst precursor, which is another aspect of this disclosure, comprises a first precursor component having the following formula (F-PM-1), a second precursor component having the following formula (F-PM-2), or a mixture or combination of both the first precursor component and the second precursor component:

M^{b}_{q-p}(M^{a}L_{q})ₘ (F-PM-1)

M^{b}Lⱼ (F-PM-2)

where M^{a} is a metal element in +p valency selected from manganese, iron, cobalt, and combinations and mixtures of two or more thereof at any proportion, L, the same or different at each occurrence, is a ligand selected from CN⁻, OCN⁻, and SCN⁻, in (F-PM-1), M^{a} complexes with q units of L on average to form a complex anion in p-q average valency, M_{b} is a metal element selected from aluminum, gallium, indium, thallium, iron, cobalt, chromium, manganese, scandium, yttrium, the lanthanide series, the actinide series, and any combination or mixture of two or more thereof at any proportion, providing a cation in +m valency, where j is an integer or non-integer, and m−1≤j≤m, m is 2, 3, 4, 5, or 6, p is 2, 3, 4, or 5, q is an integer or non-integer, and 2≤q≤6.

In specific embodiments, the first precursor component having formula (F-PM-1) is a solid insoluble in deionized water at room temperature, and/or the second precursor component having formula (F-PM-2) is a solid insoluble in deionized water at room temperature. The solids of the first precursor component and/or the second precursor component may be present in the form of solid particles or solid in gels comprising the solid and solvent. For the purpose of this disclosure, a gel is regarded as a dispersion comprising insoluble solid.

In specific embodiments, the first precursor component having formula (F-PM-1) wherein m=q−p, in which case formula (F-PM-1) can be simplified as M^{b}(M^{a}L_{q}). In one example of such embodiments, m=q−p=3, q=6, and p=3, in which case formula (F-PM-1) can be simplified as M^{b}(M^{a}L₆). Specific, non-limiting examples of the first precursor component include: ME(III)[Fe(III)(CN)₆], ME(III)[Fe(III)(OCN)₆], ME(III)[Fe(III)(SCN)₆], ME(III)[Fe(II)(CN)₅], ME(III)[Fe(II)(OCN)₅], ME(III)[Fe(II)(SCN)₅], ME(III)[Co(III)(CN)₆], ME(III)[Co(III)(OCN)₆], ME(III)[Co(III)(SCN)₆], ME(III)[Co(II)(CN)₅], ME(III)[Co(II)(OCN)₅], and ME(III)[Co(II)(SCN)₅], where ME is a metal element selected from scandium, yttrium, cobalt, manganese, iron, aluminum, gallium, indium, the lanthanides, and the actinides.

The catalyst precursor comprising the first precursor component having formula (F-PM-1) and/or a second precursor component having formula (F-PM-2) may represent an ionic compound having formula (F-PM-1) wherein each M^{b} metal atom is bonded with q units of ligand L, which are not bonded with any other metal atom and/or an ionic compound having formula (F-PM-2) wherein each M^{a} and M^{b} metal atom is bonded with m units of ligands L, which are not bonded with any other metal atom.

The first precursor component having formula (F-PM-1) may represent an ionic network wherein one M^{a} metal atom is bonded with, on average, q units of ligands, at least some of which can be bonded with another metal atom. Such ligands capable of bonding with only one metal atom is called mono-dentate, capable of bonding with two metal atoms are called bidentate ligands, and such ligands capable of bonding with three metal atoms tridentate ligands. The number q in the formula (F-PM-1) representing an ionic network can be an integer or a non-integer. The ionic network is desirably insoluable in water at room temperature and one atmospheric pressure. Given that the CN⁻, OCN⁻ and SCN⁻ ligands are bidentate, such first precursor component comprising CN⁻, OCN⁻ and/or SCN⁻ ligands can form a network solid having a formula (F-PM-1) where the number q can be a non-integer instead of an integer. Such network solid can be dispersed in a solvent such as water to form a gel. Desirably the negative charges of the ligands in the network are balanced by the positive charges of the M^{a} cations, forming an electrically neutral network, which can be in the form of a gel dispersed in a solvent such as water.

Likewise, the second precursor component having formula (F-PM-2) may represent an ionic network wherein one M^{b} metal atom is bonded with, on average, j units of ligands, where j can be any number from m-1 to less than m. At least some of the bidentatate ligands can be bonded with another metal atom. Desirably the negative charges of the ligands in the network are balanced by the positive charges of the M^{b} cations, forming an electrically neutral network, which can be in the form of a gel dispersed in a solvent such as water.

A mixture of the first precursor component having formula (F-PM-1) and a second precursor component having formula (F-PM-2) can form an interconnected ionic network wherein M^{a} and M^{b} atoms are bonded, on average, q units of ligands and j units of ligands, respectively. Desirably the negative charges of the ligands in the network are balanced by the positive charges of the M^{a} and M^{b} cations, forming an electrically neutral network, which can be in the form of a gel dispersed in a solvent such as water.

It is possible that the ionic network described above present in the catalyst precursor may comprises M^{a} and M^{b} cations not completely balanced electrically with the ligands bonded with them in certain locations in the network. In such case, additional cations, such as alkali metal ions, an ammonium ion, a proton, and the like, may be entrained in the network to electrically balance the network.

### I.3 Processes for Making Catalyst Precursors

Another aspect of this disclosure relates to a process for making a catalyst precursor, such as a catalyst precursor as described above as an aspect of this disclosure. The process comprises:

(i) providing a first material comprising a first compound having the following formula (F-I-1), and/or a second compound having the following formula (F-1-2), or a mixture of the first compound and the second compound:

M^{d}_{q-p}(M^{a}L_{q})ₖ (F-I-1)

M^{e}Lₓ (F-I-2)

where M^{a} is a metal element in +p valency selected from manganese, iron, cobalt, and combinations and mixtures of two or more thereof at any proportion, L, the same or different at each occurrence, is a ligand selected from CN⁻, OCN⁻, and SCN⁻, M^{a} complexes with q units of L on average to form a complex anion in p−q average valency, M^{d} is a metal element or a group providing a cation in +k valency, and M^{e} is a metal element or a group providing a cation in +x valency, where p is 2, 3, 4, or 5, 2≤q≤6, k is 1, 2, 3, 4, 5, or 6, and x is 1, 2, 3, 4, 5, or 6;

(ii) providing a second material having the following formula (F-II):

M^{b}ₙAₘ (F-II)

where M^{b} is a metal element in +m valency selected from aluminum, gallium, indium, thallium, iron, cobalt, chromium, manganese, scandium, yttrium, the lanthanide series, the actinide series, and any combination or mixture of two or more thereof at any proportion, and A is an anion in −n valency, wherein A differs from the complex anion in (F-I-1), mis 2, 3, 4, 5, or 6, and n is 1, 2, 3, 4, 5, or 6; and

(iii) reacting the first material and the second material to obtain a first solid precursor comprising first precursor component having the following formula (F-PM-1), or a second precursor component having the following formula (F-PM-2), or a mixture or combination of both the first precursor component and the second precursor component:

M^{b}_{q-p}(M^{a}L_{q})ₘ (F-PM-1)

M^{b}Lⱼ (F-PM-2)

where j is an integer or non-integer, and m−1≤j≤m.

In particularly advantageous embodiments, the first compound and the second compound are water soluble at a temperature in the range from 20 to 80° C., preferably water soluble at room temperature. In these embodiments, desirably M^{d} and M^{e} independently provide an alkali metal ion, a proton, or an ammonium group in (F-I-1) and (F-I-2), respectively. It is possible to mix the first material and/or the second materials in solid form to produce the first solid precursor without the need of the use of a solvent. Desirably, the first compound and/or the second compound are dispersed (e.g., dissolved) in a solvent such as water to form a dispersion (e.g., a solution, a suspension, and/or a colloidal system) of compounds (F-I-1) and/or (F-I-2), which is then allowed to contact and react with the second material having a formula (F-II). Water is a preferred solvent over other solvents due to its environmental friendliness and low safety risk. Such dispersion of the first compound and/or the second compound may take the form of a gel distributed in a solvent.

In the second material having formula (F-II), A can be an anion such as a NO₃⁻, a halogen anion, a CH₃COO⁻, a citric anion, and the like. Desirably, the second material having a formula (F-II) can be a water soluble compound such as a salt of metal M^{b}, e.g., a nitrate, a nitrite, a fluoride, a chloride, a bromide, a acetate, a citrate, and the like. Desirably, a liquid dispersion (such as an aqueous solution, an aqueous suspension, or an aqueous colloidal system) of a first material is combined with a liquid dispersion (such as an aqueous solution, an aqueous suspension, or an aqueous colloidal system) of a second material to produce a first solid precursor precipitating from the liquid phase and separable from the liquid phase. Alternatively, a liquid dispersion of a first material can be combined with a solid of a second material, mixed, and allowed to react to produce a first solid precursor, and vice versa.

The first precursor can comprise a solid of a compound represented by formula (F-PM-1), and/or a solid of a compound represented by formula (F-PM-2), and/or a solid of a mixture of a material represented by formula (F-PM-1) and a material represented by formula (F-PM-2).

The first precursor can comprise an ionic network having a formula (F-PM-1), and/or an ionic network having a formula (F-PM-2), substantially the same or similar to those as described above in association with the precursor material as an aspect of this disclosure. Such ionic network may present it itself as a gel dispersed in a solvent such as water.

The first precursor can comprise an interconnected ionic network having a mixture and/or combination of portions which collectively can be represented jointly by a formula (F-PM-1) and a formula (F-PM-2), substantially the same or similar to those as described above in association with the precursor material as an aspect of this disclosure.

The processes for making a catalyst precursor can further comprise a step (iv) of adding a third material comprising a metal element M^{c}, to the first solid precursor to obtain a second solid precursor. It is highly desirable that the third material is a water soluble compound of M^{c}. For example, the third material can be a nitrate, a nitrite, a chloride, a fluoride, a bromide, an acetate, a citrate, and the like, of metal M^{c}, or a mixture or combination thereof.

Step (iv) can be effected at least partly simultaneously in step (iii), wherein the first material, the second material and the third material are combined, and after step (iii), the first solid precursor is separated from a liquid phase in the liquid dispersion, and the first solid precursor carries a quantity of the third material. Additionally or alternatively, step (iv) can be effected at least partly after step (iii), and the process further comprises: (iiia) after step (iii), separating the first solid precursor from a liquid phase in the liquid dispersion; (iiib) optionally washing the separated first solid precursor using a solvent; and subsequently (iiic) impregnating the separated first solid precursor with a dispersion of the third material in a liquid. In more specific embodiments, the process can further comprise, after step (iiic), drying and/or calcining the impregnated first solid precursor to obtain the second solid precursor. Preferred liquid dispersions are aqueous dispersions comprising water as a solvent, more preferably as a sole solvent.

Owing to the unique processes for making the catalyst precursor, metals M^{a}, and/or M^{b} and/or M^{c} can be distributed substantially homogeneously in the catalytic precursor. So can the carbon and nitrogen atoms. The atoms of metals M^{a} and/or M^{b} can be directly bonded to the carbon and/or nitrogen atoms in the ligands CN⁻, SCN⁻, and/or OCN⁻. The homogeneous distribution of the metal atoms in the catalyst precursor enables the homogeneous distribution thereof in the catalytic component made from them via thermal decomposition, as described below.

It is highly advantageous that the metal carbide(s) and/or the metal nitride(s) are highly dispersed in the catalytic component. The metal carbide(s) and/or the metal nitride(s) can be substantially homogeneously distributed in the catalytic component, resulting in a highly dispersed distribution, which can contribute to a high catalytic activity of the catalytic component.

### I.4 Producing Catalytic Components and Catalyst Compositions

To obtain a catalytic component, one can further perform a step (v) to a catalyst precursor made pursuant to the description above:

(v) heating the first solid precursor and/or the second solid precursor at a temperature equal to or greater than 200° C., preferably in a range from 200 to 800° C., in the presence of an inert atmosphere for a period of at least 1 minute to obtain a catalytic component.

The inert atmosphere protecting the first solid precursor and/or the second solid precursor and the catalytic component after completion of thermal decomposition is absent of a gas that can oxidize the first solid precursor and/or the second solid precursor such as oxygen. It may be desirable that the inert atmosphere is absent of a gas that can reduce the first solid precursor and/or the second solid precursor such as hydrogen. It may be desirable that the inert atmosphere is a flowing stream of gas having a low water partial pressure. Thus, the inert atmosphere may comprise nitrogen gas, argon, helium, neon, mixtures of two or more thereof, and the like.

The first precursor and/or the second precursor material are then heated to an elevated temperature from T1 to T2° C., where T1 and T2 can be, independently, e.g., 200, 250, 300, 350, 400, 450, 500, 550, 600, 700, 750, 800, 850, 900, 950, or 1000, as long as T1<T2. Preferably T1=200, and T2=800. More preferably T1=200, and T2=600. Still more preferably T1=300, and T2=500. At such elevated temperature, the first precursor and/or the second precursor are heated for a period of at least 1 minutes under the protection of the inert atmosphere. Preferably, the heating period can range from t1 to t2 hours, where t1 and t2 can be, independently, e.g., 0.1, 0.2, 0.3, 0.4, 0.5, 0.6, 0.7, 0.8, 0.9, 1, 2, 4, 5, 6, 8, 10, 12, 14, 15, 16, 18, 20, 22, 24, 25, 26, 28, 30, 32, 34, 35, 36, 38, 40, 42, 44, 45, 46, or 48, as long as t1<t2.

The exposure of the first precursor and/or the second precursor to the elevated thermal decomposition temperature for the heating period results in thermal decomposition thereof to obtain a catalytic component. Upon completion of the heating period, the catalytic component may be used as is as a catalyst composition in a conversion reactor, or cooled down under the protection of the inert atmosphere, after which it can be combined with other components of the catalyst composition, such as a binder, a support, a co-catalyst, and the like, to make a catalyst composition.

The thus prepared catalytic component in step (v) can desirably comprise M^{a}, M^{b}, optionally M^{c}, carbon, nitrogen, and optionally sulfur, at a molar ratio of M^{b}, M^{c}, carbon, nitrogen, and sulfur to M^{a} of r1, r2, r3, r4, and r5, respectively, indicated below:

M^{b}:M^{c}:C:N:S:M^{a}=r1:r2:r3:r4:r5:1, where:

0.1≤r1≤1.5;

0≤r2≤0.5;

0<r3≤1;

0<r4≤1; and

0≤r5≤1; and

where M^{a} is selected from manganese, iron, cobalt, and combinations and mixtures of two or more thereof at any proportion, M^{b} is selected from aluminum, gallium, indium, thallium, chromium, scandium, yttrium, the lanthanide series, the actinide series, and any combination or mixture of two or more thereof at any proportion, M^{c} is selected from alkali metals, copper, silver, and any combinations or mixtures of two or more thereof at any proportion.

It may be desirable that at least a portion of the carbon in the catalytic component is present as a metal carbide of one or more of M^{a}, M^{b}, and M^{c}, and/or at least a portion of the nitrogen in the catalytic component is present as a metal nitride of one or more of M^{a}, M^{b}, and M^{c}, as determined by XRD of the catalytic component. More desirably, at least a portion of the carbon in the catalytic component is present as a metal carbide of one or more of M^{a} and M^{b}, and/or at least a portion of the nitrogen in the catalytic component is present as a metal nitride of one of more of M^{a} and M^{b}, as determined by XRD of the catalytic component. Without intending to be bound by a particular theory, it is believed the presence of the metal carbide(s) and the metal nitride(s) benefits at least in part to the catalytic effect of the catalytic component in the catalyst composition of this disclosure.

By using the process for making the catalyst precursor and the process for making the catalyst composition, one can surprisingly make a catalyst composition comprising metal carbide(s), metal nitride(s), or combination of both, at a low temperature of no higher than 800° C., by thermal decomposition of the first solid precursor and/or the second solid precursor comprising both carbon and nitrogen in the form of CN⁻, OCN⁻, and/or SCN⁻. Without intending to be bound by a particular theory, such low processing temperature is enabled by the presence of the metal(s), carbon, and nitrogen atoms in close proximity to each other in the structure of the first solid precursor and the second solid precursor.

Due to the substantially homogeneous distribution of the metal atoms M^{a} and M^{b} in the catalyst precursor, the directly bonding between the metal atoms to the carbon and/or nitrogen atoms in the ligands in the catalyst precursor, and the substantially homogeneous distribution of carbon and nitrogen atoms in the catalyst precursor, metal carbides and/or metal nitride phases of M^{a} and/or M^{b} can be formed in the thermal decomposition process to make the catalytic component, where the metals M^{a} and/or M^{b}, and the carbide/nitride phases thereof, can be substantially homogeneously distributed in the catalyst component thus made. Homogeneous distribution of metal(s) results in highly dispersed distribution thereof, large number of catalytically effective sites on the catalytic component, and high catalyst activity of the catalytic component.

The thus made catalytic component can be used as is as a catalyst composition for its intended use (e.g., converting syngas), i.e., as a bulk catalyst. The freshly thermally decomposed catalytic component may undergo chemical and/or physical changes when in contact with ambient air, including oxidation, water absorption, and the like. Therefore, it is highly desirable to conduct the thermal decomposition step (v) in a reactor the catalyst composition is intended for, such as a syngas converting reactor. Following the thermal decomposition, a feed (e.g., a feed comprising syngas) can replace the inert atmosphere used in step (v), whereupon a chemical process (e.g., a syngas conversion process) can be initiated in the presence of the catalyst composition under desirable conversion conditions.

Alternatively, after step (v), one can combine the catalytic component with a catalyst support material, a co-catalyst, or a solid diluent material, to form a catalyst composition. Suitable catalyst support materials for combining with the catalytic component were described earlier in this disclosure in connection with the catalyst composition. The combination of the support material and the catalytic component can be processed in any known catalyst forming processes, including but not limited to grinding, milling, sifting, washing, drying, calcination, and the like, to obtain a catalyst composition. The catalyst composition may be then disposed in an intended reactor to perform its intended function, such as a syngas converting reactor in a syngas converting process.

It is contemplated that prior to step (v), the first solid precursor and/or the second solid precursor may be combined with a catalyst support material to obtain a mixture thereof, which is subsequently subject to step (v). In such processes, the first solid precursor and/or the second solid precursor are desirably disposed on the internal and/or external surfaces of the support material. In the subsequent step (v), the catalyst precursor(s) thermally decompose to leave a catalytic component on the surface of the support material, to form a catalyst composition. Likewise, the subsequent step (v) can be desirably performed in a reactor where the catalyst composition is normally used, such as a syngas converting reactor. Alternatively, step (v) can be performed in a reactor other than the reactor the catalyst composition is intended for to obtain a catalyst composition comprising a support material and the catalytic component, which can be stored, shipped, and then disposed in a reactor it is intended for.

It is also contemplated that prior to step (v), the first solid precursor and/or the second solid precursor may be combined or formed with a precursor of a support material to obtain a support/catalytic component precursor mixture. Suitable precursors of various support materials can include, e.g., alkali metal aluminates, water glass, a mixture of alkali metal aluminates and water glass, a mixture of sources of a di-, tri-, and/or tetravalent metal, such as a mixture of water-soluble salts of magnesium, aluminum, and/or silicon, chlorohydrol, aluminum sulfate, or mixtures thereof. The support/catalytic component precursor mixture is subsequently subject to step (v) together, resulting in the formation of the catalytic component and the support material substantially in the same step. Likewise, the subsequent step (v) can be desirably performed in a reactor where the catalyst composition is normally used, such as a syngas converting reactor. Alternatively, step (v) can be performed in a reactor other than the reactor the catalyst composition is intended for to obtain a catalyst composition comprising a support material and the catalytic component, which can be stored, shipped, and then disposed in a reactor it is intended for.

### I.5 Processes for Making LAOs from Syngas Using the Catalyst Composition of the First Aspect

The catalyst composition of the first aspect of this disclosure can be advantageously used as a Fischer-Tropsch catalyst in a process for making LAOs from a feed comprising syngas.

The term “syngas” as used herein relates to a gaseous mixture consisting essentially of hydrogen (H₂) and carbon monoxide (CO). The syngas, which is used as a feed stream, may include up to 10 mol % of other components such as CO₂ and lower hydrocarbons (lower HC), depending on the source and the intended conversion processes. Said other components may be side-products or unconverted products obtained in the process used for producing the syngas. The syngas may contain such a low amount of molecular oxygen (O₂) so that the quantity of O₂ present does not interfere with the Fischer-Tropsch synthesis reactions and/or other conversion reactions. For example, the syngas may include not more than 1 mol % O₂, not more than 0.5 mol % O₂, or not more than 0.4 mol % O₂. The syngas may have a hydrogen (H₂) to carbon monoxide (CO) molar ratio of from r1 to r2, where r1 and r2 can be, independently, e.g., 0.3, 0.4, 0.5, 0.6, 0.7, 0.8, 0.9, 1, 1.2, 1.4, 1.5, 1.6, 1.8, 2.0, 2.2, 2.4, 2.5, 2.6, 2.8, 3.0, as long as r1<r2. The partial pressures of H₂ and CO may be adjusted by introduction of inert gas to the reaction mixture.

Syngas can be formed by reacting steam and/or oxygen with a carbonaceous material, for example, natural gas, coal, biomass, or a hydrocarbon feedstock through a reforming process in a syngas reformer. The reforming process can be based on any suitable reforming process, such as Steam Methane Reforming, Auto Thermal Reforming, or Partial Oxidation, Adiabatic Pre Reforming, or Gas Heated Reforming, or a combination thereof. Example steam and oxygen reforming processes are detailed in U.S. Pat. No. 7,485,767.

The syngas formed from steam or oxygen reforming includes hydrogen and one or more carbon oxides (CO and CO₂). The hydrogen to carbon oxide ratio of the syngas produced will vary depending on the reforming conditions used. The syngas reformer product(s) should contain H₂, CO and CO₂ in amounts and ratios which render the resulting syngas blend suitable for subsequent processing into either oxygenates comprising methanol/dimethyl ether or in Fischer-Tropsch synthesis.

It is possible to alter the ratio of components within the syngas and the absolute CO₂ content of the syngas by removing, and optionally recycling, some of the CO₂ from the syngas produced in one or more reforming processes. Several commercial technologies are available (e.g. acid gas removal towers) to recover and recycle CO₂ from syngas as produced in the reforming process. In at least one embodiment, CO₂ can be recovered from the syngas effluent from a steam reforming unit, and the recovered CO₂ can be recycled to a syngas reformer.

Suitable Fischer-Tropsch catalysis procedures may be found in: U.S. Pat. Nos. 7,485,767; 6,211,255; and 6,476,085; the relevant portions of their contents being incorporated herein by reference. The catalyst composition may be contained in a fixed bed reactor, a fluidized bed reactor, or any other suitable reactor.

The conversion conditions may include a wide range of temperatures. In at least one embodiment, the conversion conditions comprise a temperature in a range from T1 to T2° C., where T1 and T2 can be., e.g., 175, 180, 190, 200, 220, 240, 250, 260, 280, 290, 300, 320, 340, 350, as long as T1<T2.

The conversion conditions may include a wide range of pressures. In at least one embodiment, the absolute reaction pressure ranges from p1 to p2 kilopascal (“kPa”), wherein p1 and p2 can be, independently, e.g., 100, 150, 200, 250, 300, 350, 400, 450, 500, 600, 700, 800, 900, 1000, 1500, 2000, 2500, 3000, as long as p1<p2.

The conversion conditions may comprise a gas hourly space velocity from v1 to v2 hr-1, wherein v1 and v2 can be, independently, e.g., 500, 600, 700, 800, 900, 1000, 2000, 3000, 4000. 5000, 6000, 7000, 8000, 9000, 10000, as long as v1<v2.

In certain embodiments, the catalyst composition may be activated in the presence of a H2-containing atmosphere before step (I). Such activation can be performed ex-situ outside of the conversion reactor before being loaded into the conversion reactor, or alternatively or additionally, in-situ inside the conversion reactor. Such H2-containing atmosphere can be, e.g., syngas, high-purity hydrogen, H2/inert gas mixture, and mixtures thereof. Inert gas can be, e.g., N₂, He, Ne, Ar, and Kr. The activation may be performed at a temperature from, e.g., T1 to T2° C., where T1 and T2 can be, independently, e.g., 150, 160, 170, 180, 190, 200, 220, 240, 250, 260, 280, 300, 320, 340, 350, 360, 380, 400, 420, 440, 450, 460, 480, 500, as long as T1<T2.

In certain embodiments of the process of the first aspect of this disclosure, in step (I), the conversion product mixture comprises LAOs, in aggregate, from c1 to c2 mol %, based on the total moles of the reaction product mixture, where c1 and c2 can be, independently, e.g., 40, 45, 50, 55, 60, 65, 70, 75, 80, 85, 90, 91, 92, 93, 94, 95, as long as c1<c2. Such LAOs can advantageously comprise 1-butene, 1-pentene, 1-hexene, 1-heptene, and 1-octene.

In certain embodiments of the process of the first aspect of this disclosure, in step (I), at least one of the following is met:

(i) the conversion product mixture comprises 1-butene from c41 to c42 mol %, based on the total moles of the C4 compounds in the conversion product mixture, where c41 and c42 can be, independently, e.g., 40, 45, 50, 55, 60, 65, 70, 75, 80, 85, 90, 91, 92, 93, 94, 95, as long as c41<c42. Preferably c41=60. Preferably c41=80;
(ii) the conversion product mixture comprises 1-pentene from c51 to c52 mol %, based on the total moles of the C5 compounds in the conversion product mixture, where c51 and c52 can be, independently, e.g., 40, 45, 50, 55, 60, 65, 70, 75, 80, 85, 90, 91, 92, 93, 94, 95, as long as c51<c52. Preferably c51=50. Preferably c51=70;
(iii) the conversion product mixture comprises 1-hexene from c61 to C62 mol %, based on the total moles of the C6 compounds in the conversion product mixture, where c61 and c62 can be, independently, e.g., 40, 45, 50, 55, 60, 65, 70, 75, 80, 85, 90, 91, 92, 93, 94, 95, as long as c61<c62. Preferably c61=50. Preferably c61=70;
(iv) the conversion product mixture comprises 1-heptene from c71 to c72 mol %, based on the total moles of the C7 compounds in the conversion product mixture, where c71 and c72 can be, independently, e.g., 40, 45, 50, 55, 60, 65, 70, 75, 80, 85, 90, 91, 92, 93, 94, 95, as long as c71<c72. Preferably c71=50. Preferably c51=60;
and

(v) the conversion product mixture comprises 1-octene from c81 to c82 mol %, based on the total moles of the C8 compounds in the conversion product mixture, where c81 and c82 can be, independently, e.g., 40, 45, 50, 55, 60, 65, 70, 75, 80, 85, 90, 91, 92, 93, 94, 95, as long as c81<c82. Preferably c81=50. Preferably c81=60.

In certain embodiments of the process of the first aspect of this disclosure, in step (I), at least one of the following is met:

(i) the conversion product mixture comprises n-butane from c4-1 to c4-2 mol %, based on the total moles of the C4 compounds in the conversion product mixture, where c4-1 and c4-2 can be, independently, e.g., 5, 10, 15, 20, 25, 30, 35, 40, 45, 50, as long as c4-1<c4-2. Preferably c4-2=40. Preferably c4-2=30;

(ii) the conversion product mixture comprises n-pentane from c5-1 to c5-2 mol %, based on the total moles of the C5 compounds in the conversion product mixture, where c5-1 and c5-2 can be, independently, e.g., 5, 10, 15, 20, 25, 30, 35, 40, 45, 50, as long as c5-1<c5-2. Preferably c5-2=40. Preferably c5-2=30;

(iii) the conversion product mixture comprises n-hexene from c6-1 to C6-2 mol %, based on the total moles of the C6 compounds in the conversion product mixture, where c6-1 and c6-2 can be, independently, e.g., 5, 10, 15, 20, 25, 30, 35, 40, 45, 50, as long as c6-1<c6-2. Preferably c6-2=40. Preferably c6-2=30;

(iv) the conversion product mixture comprises n-heptane from c7-1 to c7-2 mol %, based on the total moles of the C7 compounds in the conversion product mixture, where c7-1 and c7-2 can be, independently, e.g., 5, 10, 15, 20, 25, 30, 35, 40, 45, 50, as long as c7-1<c7-2. Preferably c7-2=40. Preferably c7-2=30; and

(v) the conversion product mixture comprises n-octane from c8-1 to c8-2 mol %, based on the total moles of the C8 compounds in the conversion product mixture, where c8-1 and c8-2 can be, independently, e.g., 5, 10, 15, 20, 25, 30, 35, 40, 45, 50, as long as c8-1<c8-2. Preferably c8-2=40. Preferably c8-2=30.

In certain embodiments of the process of the first aspect of this disclosure, in step (I), at least one of the following is met, resulting in the production of internal olefins at low concentrations in the conversion product mixture:

(i) the conversion product mixture comprises 2-butene from c4-a to c4-b mol %, based on the total moles of the C4 compounds in the conversion product mixture, where c4-a and c4-2 can be, independently, e.g., 0, 2, 4, 5, 6, 8, 10, 12, 14, 15, 16, 18, 20, as long as c4-a<c4-b. Preferably c4-b=15. Preferably c4-5=10;

(ii) the conversion product mixture comprises C5 internal olefins from c5-a to c5-b mol %, based on the total moles of the C5 compounds in the conversion product mixture, where c5-a and c5-2 can be, independently, e.g., 0, 2, 4, 5, 6, 8, 10, 12, 14, 15, 16, 18, 20, as long as c5-a<c5-b. Preferably c5-b=15. Preferably c5-b=10;

(iii) the conversion product mixture comprises C6 internal olefins from c6-a to C6-b mol %, based on the total moles of the C6 compounds in the conversion product mixture, where c6-a and c6-2 can be, independently, e.g., 0, 2, 4, 5, 6, 8, 10, 12, 14, 15, 16, 18, 20, as long as c6-a<c6-b. Preferably c6-b=15. Preferably c6-b=10;

(iv) the conversion product mixture comprises C7 internal olefins from c7-a to c7-b mol %, based on the total moles of the C7 compounds in the conversion product mixture, where c7-a and c7-2 can be, independently, e.g., 0, 2, 4, 5, 6, 8, 10, 12, 14, 15, 16, 18, 20, as long as c7-a<c7-b. Preferably c7-b=15. Preferably c5-b=10; and

(v) the conversion product mixture comprises C8 internal olefins from c8-a to c8-b mol %, based on the total moles of the C8 compounds in the conversion product mixture, where c8-a and c8-2 can be, independently, e.g., 0, 2, 4, 5, 6, 8, 10, 12, 14, 15, 16, 18, 20, as long as c8-a<c8-b. Preferably c8-b=15. Preferably c8-b=10.

In certain embodiments of the process of the first aspect of this disclosure, in step (I), at least one of the following is met, resulting in the production of linear 1-alchols at low concentrations in the conversion product mixture:

(i) the conversion product mixture comprises 1-butanol from c4-m to c4-n mol %, based on the total moles of the C4 compounds in the conversion product mixture, where c4-m and c4-2 can be, independently, e.g., 0, 0.1, 0.2, 0.2, 0.3, 0.4, 0.5, 0.6, 0.7, 0.8, 0.9, 1, 1.5, 2, 2.5, 3, 3.5, 4, 4.5, 5, as long as c4-m<c4-n;

(ii) the conversion product mixture comprises 1-pentanol from c5-m to c5-n mol %, based on the total moles of the C5 compounds in the conversion product mixture, where c5-m and c5-2 can be, independently, e.g., 0, 0.1, 0.2, 0.2, 0.3, 0.4, 0.5, 0.6, 0.7, 0.8, 0.9, 1, 1.5, 2, 2.5, 3, 3.5, 4, 4.5, 5, as long as c5-m<c5-n;

(iii) the conversion product mixture comprises 1-hexanol from c6-m to C6-n mol %, based on the total moles of the C6 compounds in the conversion product mixture, where c6-m and c6-2 can be, independently, e.g., 0, 0.1, 0.2, 0.2, 0.3, 0.4, 0.5, 0.6, 0.7, 0.8, 0.9, 1, 1.5, 2, 2.5, 3, 3.5, 4, 4.5, 5, as long as c6-m<c6-n;

(iv) the conversion product mixture comprises 1-heptanol from c7-m to c7-n mol %, based on the total moles of the C7 compounds in the conversion product mixture, where c7-m and c7-2 can be, independently, e.g., 0, 0.1, 0.2, 0.2, 0.3, 0.4, 0.5, 0.6, 0.7, 0.8, 0.9, 1, 1.5, 2, 2.5, 3, 3.5, 4, 4.5, 5, as long as c7-m<c7-n; and

(v) the conversion product mixture comprises 1-octanol from c8-m to c8-n mol %, based on the total moles of the C8 compounds in the conversion product mixture, where c8-m and c8-2 can be, independently, e.g., 0, 0.1, 0.2, 0.2, 0.3, 0.4, 0.5, 0.6, 0.7, 0.8, 0.9, 1, 1.5, 2, 2.5, 3, 3.5, 4, 4.5, 5, as long as c8-m<c8-n.

One or more LAO products can be produced by separating the conversion product mixture. Any suitable separating processes and equipment maybe used, including but not limited to flashing, distillation, membrane separation, absorption, adsorption, cryogenic separation, crystallization, and the like.

### I.6 EXAMPLES

### Part I-A. Catalyst Synthesis and XRD Characterization

### Example I-A1: Preparation of La(III)[Co(III)(CN)₆] and Similar Bimetallic Catalyst Precursors

In a 500 cc flask containing 200 cc water, potassium hexacyanocobaltate(III) was dissolved (100 mmol, 33.4 g). To this solution, a trivalent metal nitrate (e.g., 100 mmol, 43.3 g La(NO₃)₃·6H₂O) was added. The initial clear solution turned into a viscous slurry upon stirring at 25-50° C. for 2-10 hrs. The reaction product was filtered and washed using deionized water to yield of ˜35 g solid. The solid was dried and used as a catalyst precursor. The reaction can be illustrated below:

La(NO₃)₃+K₃[Co(III)(CN)₆]→La[Co(III)(CN)₆↓+3KNO₃

The solid catalyst precursor was then thermally decomposed under of a flowing stream of nitrogen at a temperature between 300 and 500° C. to obtain a catalyst component. The thus made catalytic component can be analyzed by XRD to identify the respective phases.

Similarly, lanthanide cyanoferrate can be made:

La(NO₃)₃+K₃[Fe(III)(CN)₆]→La[Fe(III)(CN)₆↓+3KNO₃

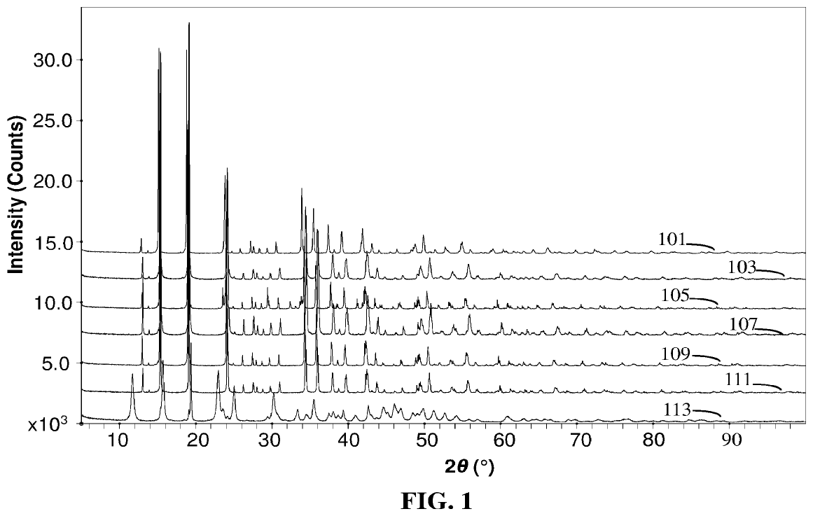

Multiple catalytic components comprising two metal elements—iron and a lanthanide—were made according to the procedure of this Example A1. The XRD analysis results of the catalyst precursors after drying are included in FIG. 1, and their solid precursor compositions are listed in TABLE I below:

TABLE I

| Reference No. | Solid Catalyst Precursor |
|---|---|
| 101 | Nd(III)[Fe(III)(CN)₆] |
| 103 | Ho(III)[Fe(III)(CN)₆] |
| 105 | Gd(III)[Fe(III)(CN)₆] |
| 107 | Er(III)[Fe(III)(CN)₆] |
| 109 | Tb(III)[Fe(III)(CN)₆] |
| 111 | Dy(III)[Fe(III)(CN)₆] |
| 113 | Y(III)[Fe(III)(CN)₆] |

### Example I-A2: Preparation of Co—La—Mn and Similar Trimetallic Catalyst Precursors

In a 500 cc flask containing 200 cc water, potassium hexacyanocobaltate(III) was dissolved (100 mmol, 33.4 g). To this solution, 8.5 g (56 mmol) of manganese sulfate was added followed by addition of a trivalent metal nitrate (e.g., 100 mmol, 43.3 g La(NO₃)₃·6H₂O) and 3.8 g (50 mmol) of NH₄SCN. The initial clear solution turned into a viscous slurry upon stirring at 25-50° C. for 2-10 hrs. The reaction product was filtered to yield of ˜45 g solid. The solid was water washed, dried and then used as catalyst precursor. The solid is believed to be a mixture comprising the following, where L, the same or different at each occurrence, is CN⁻ or SCN⁻, and x can be, independently, any integer or non-integer number ranging from 2 to 6: La[CoLₓ]; La[MnLₓ]; Mn[CoLₓ]; Co[MnLₓ]; La(SCN)₃; Mn(SCN)₂; and La(SCN)₃.

The solid catalyst precursor may be an ionic network wherein some of the CN⁻ and SCN⁻ bi-dentate ligand complex with two metal ions. Where there are defects in the network, some NH₄⁺ or K⁺ ions may be present.

The water soluble products in the above reaction can include: K₂SO_{4;} (NH₄)₂SO₄; KNH₄SO₄; KCN; KSCN; NH₄CN; and NH₄SCN, which were removed from the solid catalyst precursor by water washing.

The dried solid catalyst precursor was used in Example B1 below in an exemplary syngas conversion process, where it was studied for its catalytic effect.

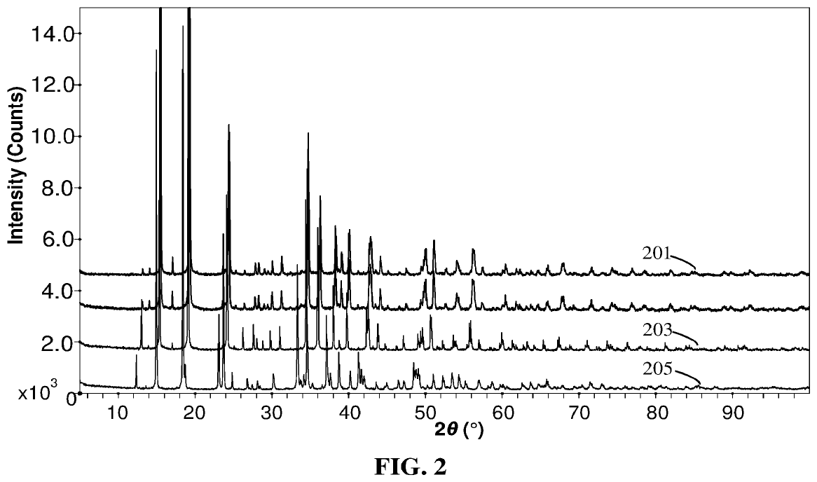

Multiple trimetallic catalytic components of this disclosure were made using the same procedure of this Example A2 by replacing La(NO₃)₃ with another lanthanide nitrate. XRD diagrams of several catalyst precursors thus made are included in FIG. 2, and elements believed to be present in the respective solid catalyst precursors are listed in TABLE II below:

TABLE II

| Reference No. | Elements in Solid Catalyst Precursor |
|---|---|
| 201 | Y, Co, Mn, C, S |
| 203 | Gd, Co, Mn, C, S |
| 205 | La, Co, Mn, C, S |

The dried solid catalyst precursor was then thermally decomposed at a temperature around 450° C., to obtain a catalytic component. XRD of the catalytic component showed cobalt 17.08 wt %, manganese 9.193 wt %, lanthanum 33.89 wt %, and sulfur 4.928 wt %, based on the total weight of the catalytic component.

Another catalytic component made by the procedure of this Example A2 comprising cobalt, manganese, and yttrium was found to comprise yttrium 28.5 wt %, cobalt 23.7 wt %, manganese 13 wt %, and Sulfur 2.2 wt %, based on the total weight of the catalytic component, according to the XRD diagram. The solid catalyst precursor to this catalytic component was used in Example B2 below in an exemplary syngas conversion process, where it was studied for its catalytic effect.

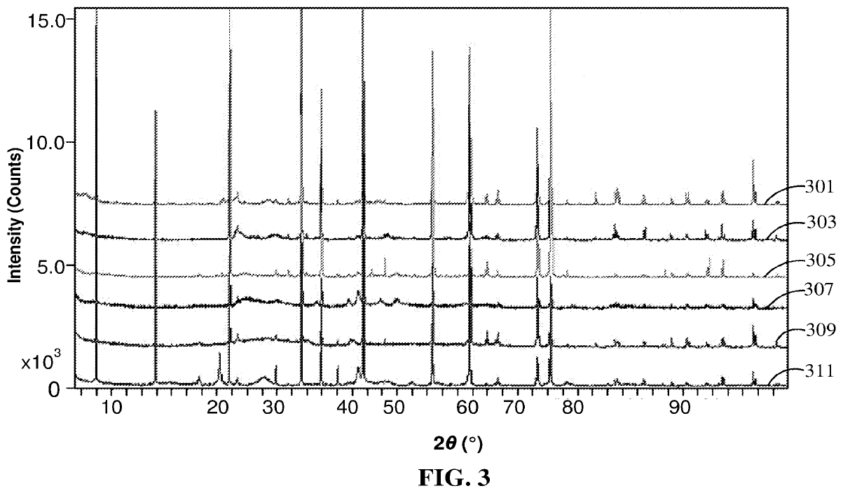

A series of bimetallic or trimetallic catalyst components of this disclosure were made using the procedures of Example A1 or A2, and tested in syngas conversion processes. Upon testing, some of the used catalytic components were then evaluated by XRD. XRD patterns of six of them are presented in FIG. 3. Metals contained in these catalytic components are listed in TABLE III below:

TABLE III

| Reference No. | Metal Elements in Catalytic Component |
|---|---|
| 301 | Pr, Co, Cu |
| 303 | Gd, Co, Cu |
| 305 | Y, Co, Mn |
| 307 | Y, Co, Cu |
| 309 | Er, Fe |
| 311 | La, Co |

Thermogravimetric analysis of a catalyst precursor of an embodiment of this disclosure comprising holmium hexacyanoferrate (Ho(III)[Fe(III)(CN)₆]) was conducted twice at a temperature elevation rate of 10° C./min under air purge. The analysis results are shown in

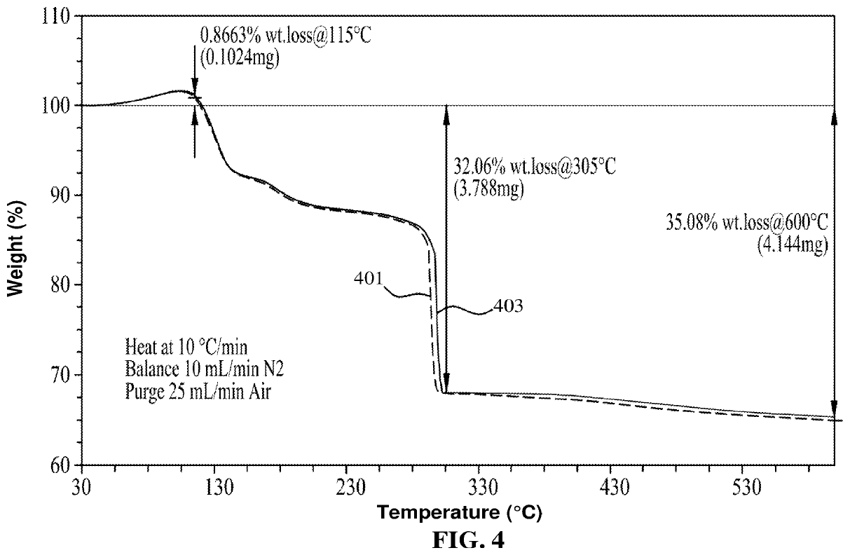

FIG. 4 as weight-temperature curves. The dotted curve 401 shows analysis result of a first sample taken from the catalyst precursor. The solid curve 403 shows analysis result of a second sample taken from the same catalyst precursor, but analyzed the day the first sample was analyzed. As can be seen from FIG. 4, the two curves align with each other very closely, indicating the precursor material did not undergo significant change overnight. The curves clearly show that around 305° C., significant change occurred to the precursor material resulting in total weight loss of 32.06%, indicating thermal decomposition of the precursor.

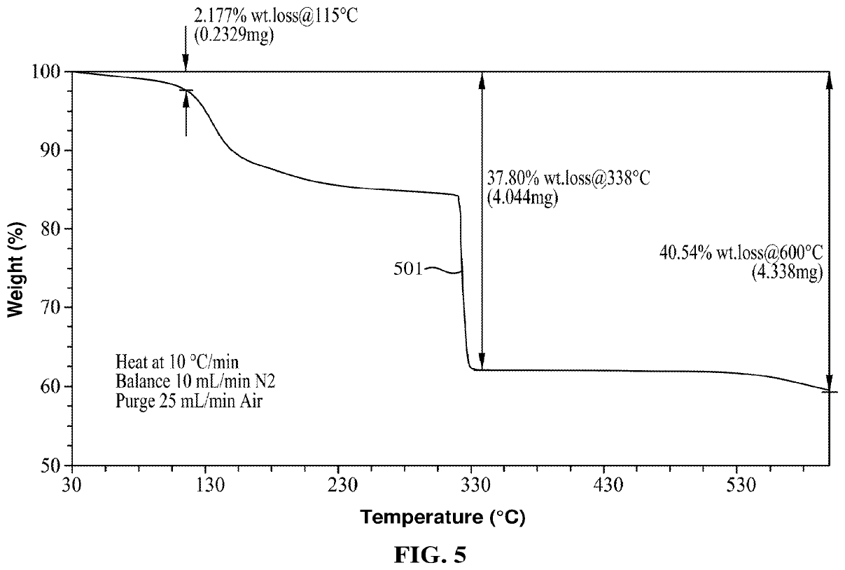

Similarly, FIG. 5 shows a thermogravimetric analysis result of a catalyst precursor 501 of another embodiment of this disclosure comprising gadolinium hexacyanocobaltate (Gd(III)(Co(III)(CN)₆) at a temperature elevation rate of 10° C./min under air purge. Around 338° C., significant changes occurred to the catalyst precursor, resulting in a total weight loss of 37.80%, indicating thermal decomposition of the catalyst precursor.

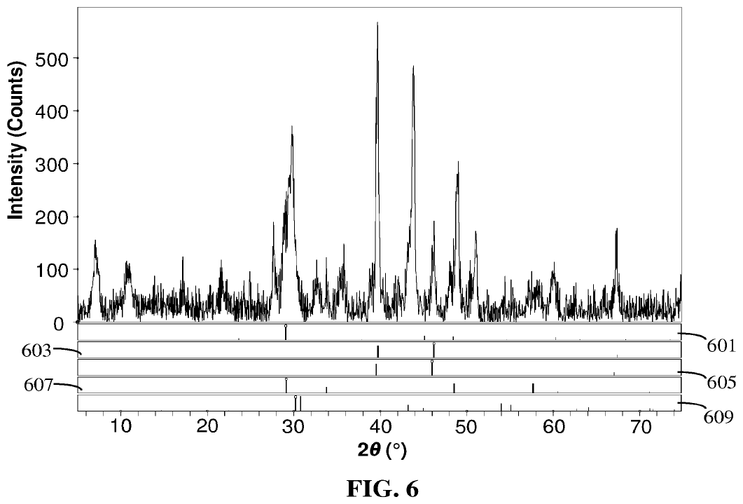

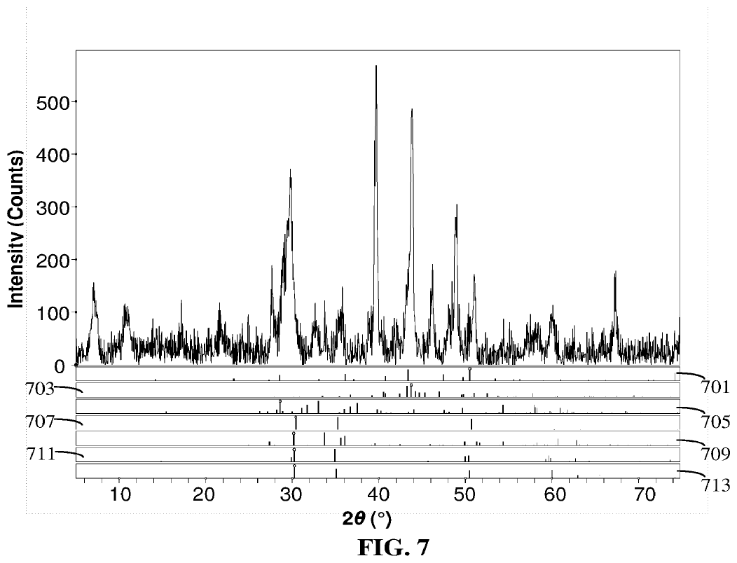

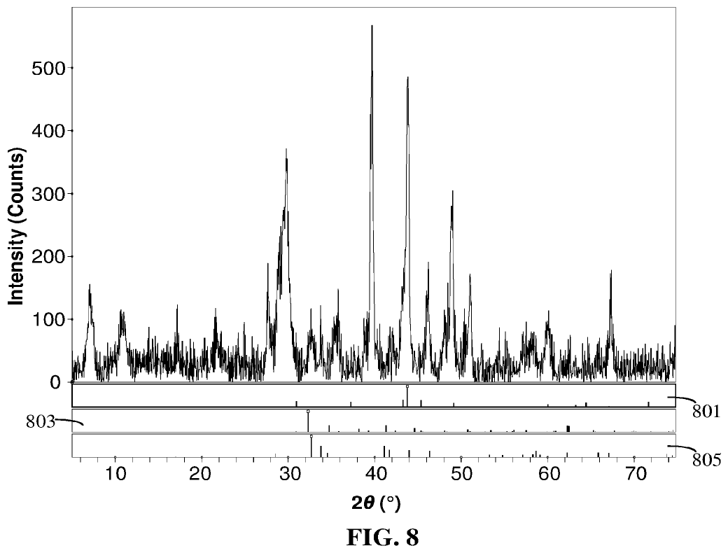

A sample of a catalyst component comprising cobalt, manganese, and yttrium prepared pursuant to the procedure of this Example A2 was characterized by powder XRD. The XRD diagram and the various peak groups identified by reference numerals are provided in FIGS. 6, 7, and 8. These peak groups match with known peaks of various phases pursuant to International Center for Diffraction Data (“ICDD”) XRD peak library as listed in TABLE IV below. The peak groups for carbon (01-079-1473), carbon nitride (01-078-1747, C₃N₄), yttrium (01-089-9233), and yttrium manganese (Mn₂Y, 03-066-0003), identifiable from the XRD graph, are not provided in FIGS. 6, 7, and 8.

Clearly in the catalytic component, multiple phases of carbides of several metals and multiple phases of nitrides of several metals are present as evidenced by the XRD diagram. Without intending to be bound by a particular theory, it is believed that the presence of these carbides and/or nitride phases are conducive to the catalytic activity of the catalytic component, especially for the purpose of converting syngas.

In the catalytic component, metal and metal alloy phases are also present. These phases may perform catalytic effect as well.

TABLE IV

| Peak Group in | ICDD Library | | Phase |
|---|---|---|---|
| XRD | Identification No. | Phase Name | Composition |
| — | 01-079-1473 | Carbon | C |
| — | 01-078-1747 | Carbon Nitride | C₃N₄ |
| — | 01-089-2933 | Yttrium | Y |
| — | 03-066-0003 | Yttrium Manganese | Mn₂Y |
| 601 | 01-078-1747 | Carbon Nitride | C₃N₂ |
| 603 | 01-083-8039 | Cobalt Nitride | CoN |
| 605 | 01-083-8037 | Manganese Nitride | MnN |
| 607 | 01-080-5705 | Yttrium Nitride | YN |
| 609 | 01-081-0300 | Manganese Nitride | Mn₃N₂ |
| 701 | 01-081-9789 | Manganese Carbide | Mn₁₁C₂ |
| 703 | 01-080-1700 | Manganese Carbide | Mn₅C₂ |
| 705 | 01-088-0569 | Yttrium Carbide | Y₄(C₂)₂C |
| 707 | 01-080-5687 | Yttrium Carbide | YC |
| 709 | 01-081-4226 | Yttrium Carbide | Y₁₅C₁₉ |
| 711 | 01-072-6343 | Yttrium Carbide | Y₂C |
| 713 | 01-073-0501 | Yttrium Carbide | YC_{0.40} |
| 801 | 03-065-8990 | Cobalt Yttrium | Co₅Y |
| 803 | 03-065-5573 | Cobalt Yttrium | Co₇Y₉ |
| 805 | 01-073-0117 | Yttrium Cobalt | YCo |

It is totally surprising that carbide phases and nitride phases were formed in the catalytic component at such low thermal decomposition temperature employed. In typical processes for making metal carbides involving the reaction of a metal source and a carbon source, one would have to run the reaction at above 800° C. to effect the formation of a metal carbide. In typical processes for making metal nitrides involving the reaction of a metal source and ammonia, the temperature required is higher than 650° C. Yet, by utilizing the processes of this disclosure involving the formation of a catalyst precursor comprising the metals, carbon, and nitrogen in the compounds and/or ionic network, one can obtain a catalyst component comprising both a metal carbide phase and a metal nitride phase at a temperature significantly lower than 800° C., such as lower than 600° C., and even lower than 500° C. Such low thermal decomposition temperature enables the in-situ thermal decomposition of the catalyst precursor to produce a catalytic component in a conversion reactor that the catalytic component is intended for, which is particularly advantageous if the conversion process is typically conducted around or lower than the thermal decomposition. Further, the metal carbide phase(s) and the metal nitride phase(s) are believed to be distributed intimately and substantially homogenously with other phases, including the individual metal phases, the mixed metal phases, the carbon phase, and the carbon nitride phases in the catalytic component, providing highly dispersed distribution thereof in the catalytic component, resulting in high catalytic activity as demonstrated by the syngas conversion process examples below. This is contrary to conventional processes for making metal carbide(s) and metal nitride(s), which tend to result in in-homogenous distribution thereof, typically more preferentially on the surface only, resulting in likely low dispersion and low catalytic activity.

### Comparative Example I-A3: Preparation of a Trimetallic Oxide Catalyst

A La—Co—Mn oxide system, compositionally similar to the catalytic component in Example B1 above in terms of La, Co, and Mn content, synthesized via aqueous phase co-precipitation of La(NO₃)₃, Co(NO₃)₂ and Mn(NO₃)₂ with Na₂CO₃ and then calcined in air at between 450-550° C. The thus made comparative catalyst composition comprises oxides and mixed oxides of La, Co, and Mn, and is believed to be free of metal carbide and metal nitride phases.

### Part I-B: Processes for Making LAOs from Syngas Using the Catalyst Component of the First Disclosure

### Example I-B1: Process for Converting Syngas Using the Co—Y—Mn Trimetallic Catalytic Component 201 of this Disclosure for Making LAOs

A 1:3 mixture of catalyst component 201 prepared in Example I-A2 above and silicon carbide (both components sized between 40-60 mesh) were loaded into a fixed-bed reactor system. The catalyst was dried at 110° C. under an N₂ purge at 15 bar pressure and GHSV=2000 h⁻¹ for 2 h. Maintaining the N₂ purge, reactor pressure, and GHSV, the reactor temperature was then increased to 400° C. at a 1° C./min ramp rate. The reactor was kept at 400° C. for 2 h and the reactor was cooled to the Fischer-Tropsch synthesis temperature (e.g., 250° C.). Once the temperature stabilized, the reactor feed was switched to a mixture of H₂ and CO. The range of conditions explored were as follows: (1) temperature, 150-350° C.; (2) pressure, 1-50 bar; (3) H₂:CO ratio, 1:3 to 3:1, and (4) GHSV, 1000-10,000 h⁻¹. Product selectivities are reported in TABLE V below.

TABLE V

| Product | Carbon number | Product Type | Selectivity (%) |
|---|---|---|---|
| 1-Butanol | 4 | Alcohol | 1.5 |
| 1-Butene | 4 | LAO | 6.4 |
| cis-2-butene | 4 | LIO | 0.3 |
| n-Butane | 4 | n-Paraffin | 2.8 |
| 1-Pentanol | 5 | Alcohol | 1.3 |
| 1-Pentene | 5 | LAO | 3.8 |
| i-Pentane | 5 | isoparaffin | 0.1 |
| n-Pentane | 5 | n-Paraffin | 1.6 |
| 1-Hexanol | 6 | Alcohol | 0.9 |
| 1-Hexene | 6 | LAO | 2.1 |
| i-Hexane | 6 | isoparaffin | 1.5 |
| n-Hexane | 6 | n-Paraffin | 1.1 |
| 1-Heptanol | 7 | Alcohol | 0.6 |
| 1-Heptene | 7 | LAO | 1.3 |
| n-Heptane | 7 | n-Paraffin | 1.2 |
| 1-Octanol | 8 | Alcohol | 0.5 |
| 1-Octene | 8 | LAO | 0.8 |
| cis-2-Octene | 8 | LIO | 0.2 |
| trans-2-Octene | 8 | LIO | 0.7 |
| i-Octane | 8 | isoparaffin | 0.7 |
| n-Octane | 8 | n-Paraffin | 0.2 |

As can be seen from TABLE V, the selectivities for C4, C5, and C6 LAOs were consistently higher than those for C4, C5, and C6 n-paraffins and C4, C6, and C6 primary alcohols, respectively, demonstrating the suitability of the catalyst component 201 for converting syngas to LAOs.

### Example I-B2: Process for Converting Syngas Using a Co—La—Mn Trimetallic Catalytic Component 205 of This Disclosure for Making LAOs

The same experiment of Example I-B1 above was carried out, except the catalyst component 205 prepared in Example I-A2 above was used in placed of catalyst component 201. Product selectivities are reported in TABLE VI below.

As can be seen from TABLE VI, the selectivities for C4, C5, and C6 LAOs were consistently higher than those for C4, C5, and C6 n-paraffins and C4, C6, and C6 primary alcohols, respectively, demonstrating the suitability of the catalyst component 205 for converting syngas to LAOs.

TABLE VI

| Product | Carbon Number | Product Type | Selectivity (%) |
|---|---|---|---|
| 1-Butanol | 4 | Alcohol | 0.8 |
| 1-Butene | 4 | LAO | 3.4 |
| cis-2-Butene | 4 | LIO | 0.2 |
| trans-2-Butene | 4 | LIO | 0.2 |
| n-Butane | 4 | n-Paraffin | 0.9 |
| 1-Pentanol | 5 | Alcohol | 0.9 |
| i-Pentene | 5 | Isoolefin | 0.1 |
| i-Pentane | 5 | Isoparaffin | 0.3 |
| 1-Pentene | 5 | LAO | 2.1 |
| cis-2-Pentene | 5 | LIO | 0.2 |
| trans-2-Pentene | 5 | LIO | 0.1 |
| n-Pentane | 5 | n-Paraffin | 0.6 |
| 1-Hexanol | 6 | Alcohol | 1.1 |
| i-Hexane | 6 | Isoparaffin | 0.4 |
| 1-Hexene | 6 | LAO | 1.3 |
| trans-2-Hexene | 6 | LIO | 0.1 |
| n-Hexane | 6 | n-Paraffin | 0.5 |
| 1-Heptanol | 7 | Alcohol | 0.8 |
| i-Heptane | 7 | Isoparaffin | 0.4 |
| 1-Heptene | 7 | LAO | 0.9 |
| cis-2-Heptene | 7 | LIO | 0.1 |
| trans-2-Heptene | 7 | LIO | 0.1 |
| 1-Octanol | 8 | Alcohol | 0.6 |
| i-Octane | 8 | Isoolefin | 0.4 |
| 1-Octene | 8 | LAO | 0.7 |
| cis-2-Ocetene | 8 | LIO | 0.1 |
| trans-2-Octene | 8 | LIO | 0.1 |
| n-Octane | 8 | n-Paraffin | 0.3 |

### II. The Second Aspect of This Disclosure

The second aspect of this disclosure generally relates to a process for converting syngas to make LAOs comprising one or more of the following steps:

(I) contacting a feed comprising syngas with a catalyst composition in a conversion reactor under conversion conditions to produce a conversion product mixture comprising a linear alpha-olefin, wherein the catalyst composition comprises:

a support; and

a plurality of nanoparticles on the support, wherein:

each nanoparticle comprises a kernel, the kernels have an average particle size from 4 to 100 nm and a particle size distribution of no greater than 20%; the kernels comprise oxygen, a metal element M1, optionally sulfur, optionally phosphorus, an optional metal element M2, and optionally a third metal element M3, where:

M1 is selected from Mn, Fe, Co, and combination of two or more thereof;

M2 is selected from Ni, Zn, Cu, Mo, W, Ag, and combinations thereof;

M3 is selected from Y, Sc, alkaline metals, the lanthanides, group 13, 14, or 15 elements, and combinations thereof; and

the molar ratios of M2, M3, S, and P, if any, to M1 is r1, r2, r3, and r4, respectively, 0≤r1≤2, 0≤r2≤2, 0≤r3≤5, and 0≤r4≤5; and

(II) obtaining a linear alpha-olefin product from the conversion product mixture.

More details of the process follow.

### II.1 Kernel Characteristics

A nanoparticle may be present as a discreet particle dispersed in a media such as a solvent, e.g., a hydrophobic solvent such as toluene in certain embodiments. Alternatively, a nanoparticle may be stacked next to a plurality of other nanoparticles in the composition of this disclosure. A nanoparticle in the nanoparticle composition of this disclosure comprises a kernel which are observable under a transmission electron microscope. The nanoparticle may in certain embodiments further comprises one or more long-chain groups attached to the surface thereof. Alternatively, a nanoparticle may consist essentially of, or consist entirely of a kernel only.

A kernel in a nanoparticle can have a largest dimension in a range of from 4 nanometers to 100 nanometers. Kernels may have a near spherical or elongated shape (e.g. rod-shaped). Kernels that are elongated may have an aspect ratio of from 1 to 50, such as from 1.5 to 30, from 2 to 20, from 2 to 10, or from 3 to 8. The aspect ratio is the length of a longer side of the kernel divided by the length of a shorter side of the kernel. For example, a rod-shaped kernel of diameter 4 nm and length of 44 nanometers has an aspect ratio of 11.

The kernels of the nanoparticles in the nanoparticle compositions of this disclosure may have a particle size distribution of 20% or less. The particle size distribution is expressed as a percentage of the standard deviation of the particle size relative to the average particle size. For example, a plurality of kernels that have an average size of 10 nanometers and a standard deviation of 1.5 nanometers has a particle size distribution of 15%. The kernels of the nanoparticles in the nanoparticle compositions of this disclosure may have an average particle size of from 4 to 100 nm, such as 4 to 35 nm, or 4 to 20 nm.

Particle size distribution is determined by Transmission Electron Microscopy (“TEM”) measurement of nanoparticles deposited on a flat solid surface.

The kernels of the nanoparticles in the nanoparticle compositions of this disclosure may be crystalline, semi-crystalline, or amorphous in nature.

Kernels are composed of at least one metal element. The at least one metal may be selected from groups Mn, Fe, Co, Zn, Cu, Mo, W, Ag, Y, Sc, alkaline metals, the lanthanide series, group 13, 14, and 15, and combinations thereof. Where the at least one metal element comprises two or more metals, the metals may be designated as M1, M2, and M3, according to the number of metal elements. M1 may be selected from manganese, iron, cobalt, combinations of iron and cobalt at any proportion, combinations of iron and manganese at any proportion, combinations of cobalt with manganese at any proportion, and combinations of iron, cobalt, and manganese at any proportion. In specific embodiments, M1 is a single metal of manganese, cobalt, or iron. Where M1 comprises a binary mixture/combination of cobalt and manganese, cobalt may be present at a higher molar proportion than manganese. Where M1 comprises a binary mixture/combination of iron and manganese, iron may be present at a higher molar proportion than manganese. Without intending to be bound by a particular theory, it is believed that the presence of M1 provides at least a portion of the catalytic effect of the catalyst composition of the third aspect of this disclosure.

M2 may be selected from nickel, zinc, copper, molybdenum, tungsten, silver, and combinations thereof. Without intending to be bound by a particular theory, it is believed that the presence of M2 promotes the catalytic effect of M1 in the supported nanoparticle compositions of the first aspect of this disclosure.

The presence of M3 in the compositions of this disclosure is optional. If present, M3 may be selected from Y, Sc, lanthanides, and metal elements of Groups 1, 13, 14, or 15, and any combination(s) and mixture(s) of two or more thereof at any proportion. In certain embodiments, M3 is selected from aluminum, gallium, indium, thallium, scandium, yttrium, and the lanthanide series, and combination thereof. In some embodiments, M3 is selected from gallium, indium, scandium, yttrium, and lanthanides, and combinations thereof. Preferred lanthanide are: La, Ce, Pr, Nd, Gb, Dy, Ho, Er, and combinations thereof. Without intending to be bound by a particular theory, it is believed the presence of metal M3 can promote the catalyst effect of the catalyst compositions of the third aspect of this disclosure.

The kernels can further comprise oxygen in the form of, e.g., a metal oxide. The presence of a metal oxide can be indicated by the XRD graph of the catalyst composition. By a “metal oxide,” it is meant to include oxide of a single metal, or a combination of two or more metals M1, M2, and/or M3. Suitably the kernel may comprise an oxide of a single metal, or a combination of two or more metals of M1, and/or M2. Suitably the kernel may comprise an oxide of a single metal, or a combination of two or more metals of M1. In at least one embodiment, the catalytic component may comprise one or more of iron oxide, cobalt oxide, manganese oxide, (mixed iron cobalt) oxide, (mixed iron manganese) oxide, mixed (cobalt manganese) oxide, and mixed (cobalt, iron, and manganese) oxide. In at least one embodiment, the kernel may comprise an oxide of a single metal, or a combination of two or more metals of M2 (e.g., yttrium and the lanthanides). The kernel may comprise an oxide of a metal mixture comprising an M1 metal and an M2 metal. The identification of the presence of an oxide phase in a nanoparticle can be conducted by comparing the XRD data of the nanoparticle against an XRD peak database of oxides, such as those available from International Center for Diffraction Data (“ICDD”).

The kernel compositions of this disclosure may optionally comprise sulfur. Without intending to be bound by a particular theory, in certain embodiments, the presence of sulfur can promote the catalytic effect of the catalyst composition created from the nanoparticle compositions comprising kernels. The sulfur may be present as a sulfide, sulfate, or other sulfur-containing compound of one or more metals of M1, M2, and/or M3.

The kernel compositions of this disclosure may optionally comprise phosphorus. Without intending to be bound by a particular theory, in certain embodiments, the presence of phosphorus can promote the catalytic effect of the catalyst composition created from the nanoparticle compositions comprising kernels. The phosphorus may be present as a phosphide of one or more metals of M1, M2, and/or M3.

In specific embodiments, the kernel of a nanoparticle composition of this disclosure consists essentially of M1, M2, M3, oxygen, optionally sulfur, and optionally phosphorus e.g., comprising ≥85, or ≥90, or ≥95, or ≥98, or even ≥99 wt % of M1, M2, M3, oxygen, optionally sulfur, and optionally phosphorus based on the total weight of the kernel.

The molar ratios of M2 to M1 (“r1”), M3 to M1 (“12”), oxygen to M1 (“r3”), sulfur to M1 (“r4”), and phosphorus to M1 (“r5”), in the kernel of a nanoparticle composition of this disclosure are calculated from the aggregate molar amounts of the elements in question. Thus, if M1 is a combination/mixture of two or more metals, the aggregate molar amount of all metals of M1 is used for calculating the ratios. If M2 is a combination/mixture of two or more metals, the aggregate molar amounts of all metals M2 is used for calculating the ratio r1. If M3 is a combination/mixture of two or more metals, the aggregate molar amounts of all metals M3 is used for calculating the ratio r2.

The molar ratio of M2 to M1 in the kernel of a nanoparticle composition of this disclosure, r1, can be from r1a to r1b, where r1a and r1b can be, independently, e.g., 0, 0.05, 0.1, 0.2, 0.3, 0.4, 0.5, 0.6, 0.7, 0.8, 0.9, 1, 1.1, 1.2, 1.3, 1.4 or 1.5, as long as r1a<r1b. In some embodiments, r1a=0, r1b=2; such as r1a=0, r1b=0.5; or r1a=0.05, r1b=0.5. In at least one embodiment, r1 is in the vicinity of 0.5 (e.g., from 0.45 to 0.55), meaning that M1 is present in the kernel at substantially twice the molar amount of M2.

The molar ratio of M3 to M1 in the kernel of a nanoparticle compositions of this disclosure, r2, can be from r2a to r2b, where r2a and r2b can be, independently, e.g., 0, 0.005, 0.01, 0.05, 0.1, 0.2, 0.3, 0.4, or 0.5, as long as r2a<r2b. In some embodiments, r2a=0, r2b=5; such as r2a=0.005, r2b=0.5.Thus M3, if present, is at a substantially lower molar amount than M1.

The molar ratio of oxygen to M1 in the kernel of a nanoparticle composition of this disclosure, r3, can be from r3a to r3b, where r3a and r3b can be, independently, e.g., 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5, as long as r3a<r3b. In some embodiments, r3a=0.05, r3b=5; such as r3a=0.5, r3b=4; or r3a=1, r3b=3.

The molar ratio of sulfur to M1 in the kernel of a nanoparticle composition of this disclosure, r4, can be from r4a to r4b, where r4a and r4b can be, independently, e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5, as long as r4a<r4b. In some embodiments, r4a=0, r4b=5; such as r4a=0, r4b=2.

The molar ratio of phosphorus to M1 in the kernel of a nanoparticle composition of this disclosure, r5, can be from r5a to r5b, where r5a and r5b can be, independently, e.g., 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5, as long as r5a<r5b. In some embodiments, r5a=0, and r5b=5; such as r5a=0 and r5b=2.

In specific embodiments, the metal(s) M1 can be distributed substantially homogeneously in the kernel. Additionally and/or alternatively, the metal(s) M2 can be distributed substantially homogeneously in the kernel. Additionally and/or alternatively, the metal(s) M3 can be distributed substantially homogeneously in the kernel. Additionally and/or alternatively, oxygen can be distributed substantially homogeneously in the kernel. Still additionally and/or alternatively, sulfur can be distributed substantially homogeneously in the kernel. Additionally and/or alternatively, phosphorus can be distributed substantially homogeneously in the kernel.

It is highly advantageous that the metal oxide(s) are highly dispersed in the kernel. The metal oxide(s) can be substantially homogeneously distributed in the kernel, resulting in a highly dispersed distribution, which can contribute to a high catalytic activity of the catalyst composition comprising nanoparticle compositions that comprise kernels.

The nanoparticle composition of this disclosure may comprise or consist essentially of the kernel of this disclosure, e.g., comprising ≥85, or ≥90, or ≥95, or ≥98, or even ≥99 wt % of the kernel, based on the total weight of the nanoparticle composition. The nanoparticle composition of the present disclosure may comprise long-chain hydrocarbyl groups disposed on (e.g. attached to) the kernel.

### Nanoparticle Formation

The nanoparticle composition, of this disclosure may be produced from a first dispersion system at a first temperature (T1). A first dispersion system comprises a long-chain hydrocarbon solvent, a salt of at least one long-chain organic acid and the at least one metal element, optionally sulfur or an organic sulfur compound (which can be soluble in the long-chain hydrocarbon solvent), and optionally an organic phosphorus compound (which can be soluble in the long-chain hydrocarbon solvent). The salt of at least one long-chain organic acid and the at least one metal element may be formed in situ with a salt of a second organic acid and the at least one metal element, and a long-chain organic acid

The T1 may comprise temperatures from T1a to T1b, where T1a and T1b can be, independently, e.g., 0, RT, 35, 40, 45, 50, 75, 100, 125, 150, 175, 200, 225, 250, 275, or 300° C., as long as T1a<T1b, such as T1a=RT, T1b=250° C.; or T1a=35° C., T1b=150° C. The first temperature may be maintained for from 10 min to 100 hours, such as from 10 min to 10 hours, 10 minutes to 5 hours, 10 minutes to 3 hours, or 10 minutes to 2 hours. The first dispersion system may be held under inert atmosphere or under pressure reduced below atmospheric pressure. For example, the first dispersion system may be maintained under flow of nitrogen or argon, and alternatively, may be attached to a vacuum reducing the pressure to less than 760 mmHg, such as less than 400 mmHg, less than 100 mmHg, less than 50 mmHg, less than 30 mmHg, less than 20 mmHg, less than 10 mmHg, or less than 5 mmHg. The choice of maintaining the first dispersion system under flow of inert gas versus reduced pressure may affect the size of the nanoparticles produced. Without being limited by theory, it is possible that a first dispersion system under reduced pressure has fewer contaminants and byproducts than if it was maintained under flow of inert gas and the fewer contaminants may allow for formation of smaller nanoparticles. In some embodiments, maintaining the first dispersion system under reduced pressure may decrease nanoparticle size without affecting particle size distribution as compared to maintaining the first dispersion system under flow of inert gas. In other embodiments, maintaining the first dispersion system under flow of inert gas may increase nanoparticle size without affecting particle size distribution as compared to maintaining the first dispersion system under reduced pressure.

The long-chain hydrocarbon solvent may comprise saturated and unsaturated hydrocarbons, aromatic hydrocarbons, and hydrocarbon mixture(s).

Some example saturated hydrocarbons suitable for use as the long-chain hydrocarbon

solvent are C12+ hydrocarbons, such as C12 to C24, C14 to C24, C16 to C22, C16 to C20, C16 to C18 hydrocarbons, such as n-dodecane (mp −10° C., bp 214° C. to 218° C.), n-tridecane (mp −6° C., bp 232° C. to 236° C.), n-tetradecane (mp 4° C. to 6° C., bp 253° C. to 257° C.), n-pentadecane (mp 10° C. to 17° C., bp 270° C.), n-hexadecane (mp 18° C., bp 287° C.), n-heptadecane (mp 21° C. to 23° C., bp 302° C.), n-octadecane (mp 28° C. to 30° C., bp 317° C.), n-nonadecane (mp 32° C., bp 330° C.), n-icosane (mp 36° C. to 38° C., bp 343° C.), n-henicosane (mp 41° C., bp 357° C.), n-docosane (mp 42° C., bp 370° C.), n-tricosane (mp 48° C. to 50° C., bp 380° C.), n-tetracosane (mp 52° C., bp 391° C.), or mixture(s) thereof.

Some example unsaturated hydrocarbons suitable for use as the long-chain hydrocarbon solvent include C12+ unsaturated unbranched hydrocarbons, such as C12 to C24, C14 to C24, C16 to C22, C16 to C20, C16 to C18 unsaturated unbranched hydrocarbons (the double-bond may be cis or trans and located in any of the 1,2,3,4,5,6,7,8,9,10,11, or 12 positions), such as 1-dodecene (mp −35° C., bp 214° C.), 1-tridecene (mp −23° C., bp 232° C. to 233° C.), 1-tetradecene (mp −12° C., bp 252° C.), 1-pentadecene (mp −4° C., bp 268° C. to 239° C.), 1-hexadecene (mp 3° C. to 5° C., bp 274° C.), 1-heptadecene (mp 10° C. to 11° C., bp 297° C. to 300° C.), 1-octadecene (mp 14° C. to 16° C., bp 315° C.), 1-nonadecene (mp 236° C., bp 329° C.), 1-icosene (mp 26° C. to 30° C., bp 341° C.), 1-henicosene (mp 33° C., bp 353° C. to 354° C.), 1-docosene (mp 36° C. to 39° C., bp 367° C.), 1-tricosene (bp 375° C. to 376° C.), 1-tetracosene (bp 380° C. to 389° C.), trans-2-dodecene (mp −22° C., bp 211° C. to 217° C.), trans-6-tridecene (mp −11° C., bp 230° C. to 233° C.), cis-5-tridecene (mp −11° C. to -10° C., bp 230° C. to 233° C.), trans-2-tetradecene (mp 1° C. to 3° C., bp 250° C. to 253° C.), trans-9-octadecene (mp 23° C. to 25° C., bp 311° C. to 318° C.), cis-12-tetracosene (mp 96° C. to 97° C., bp 385° C. to 410° C.), or mixture(s) thereof. In some embodiments, the long-chain hydrocarbon solvent is 1-octadecene.

Aromatic hydrocarbons suitable for use as the long-chain hydrocarbon may comprise any of the above alkanes and alkenes where a hydrogen atom is substituted for a phenyl, naphthyl, anthracenyl, pyrrolyl, pyridyl, pyrazyl, pyrimidyl, imidazolyl, furanyl, or thiophenyl substituent.

Hydrocarbon mixtures suitable for use as the long-chain hydrocarbon may comprise mixtures with sufficiently high boiling points such that at least partial decomposition of the metal salts may occur upon heating below or at the boiling point of the mixture. Suitable mixtures may include: kerosene, lamp oil, gas oil, diesel, jet fuel, or marine fuel.

The long-chain organic acid may comprise any suitable organic acid with a long-chain, such as saturated carboxylic acids, mono unsaturated carboxylic acids, polyunsaturated carboxylic acids, saturated or unsaturated sulfonic acids, saturated or unsaturated sulfinic acids, saturated or unsaturated phosphonic acids, saturated or unsaturated phosphinic acids.

The long-chain organic acid may be selected from C12+ organic acids, such as C12 to C24, C14 to C24, C16 to C22, C16 to C20, or C16 to C18 organic acids. In some embodiments, the organic acid is a fatty acid, for example: caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, behenic acid, lignoceric acid, myristoleic acid, palmitoleic acid, sapienic acid, oleic acid, elaidic acid, vaccenic acid, petroselenic acid, linoleic acid, linoelaidic acid, a-linolenic acid, y-linolenic acid, stearidonic acid, gondoic acid, paullinic acid, gondoic acid, gadoleic acid, arachidonic acid, eicosenoic acid, eicosapentaenoic acid, brassidic acid, erucic acid, adrenic acid, osbond acid, clupanodonic acid, docosahexaenoic acid, nervonic acid, colneleic acid, colnelenic acid, etheroleic acid, or etherolenic acid.

The long-chain organic acid may be selected from C12+ unsaturated acids, such as C12 to C24, C14 to C24, C16 to C22, C16 to C20, C16 to C18 unsaturated acids, such as myristoleic acid, palmitoleic acid, sapienic acid, vaccenic acid, petroselenic acid, oleic acid, elaidic acid, paullinic acid, gondoic acid, gadoleic acid, eicosenoic acid, brassidic acid, erucic acid, nervonic acid.

The long-chain organic acid may be selected from myristoleic acid, palmitoleic acid, cis-vaccenic acid, paullinic acid, oleic acid, gondoic acid, or gadoleic acid. In some embodiments, the long-chain organic acid is oleic acid.

The long-chain organic acids used to prepare the metal salts may be similar in chain length to the long-chain hydrocarbon solvent, such as where the long-chain organic acid and the long-chain hydrocarbon do not differ in numbers of carbon atoms by more than 4, such as 3 or less, or 2 or less. For example, if metal oleate salts are used, then suitable long-chain hydrocarbon solvents may include: 1-heptadecene, 1-octadecene, 1-nonadecene, trans-2-octadecene, cis-9-octadecene or mixture(s) thereof.

Metal salts of the long-chain organic acid comprise the salt of (i) at least one metal selected from groups 1, 2, 3, 4, 5, 6, 11, 12, 13, 14, and 15, Mn, Fe, Co, Ni, or W, and combinations thereof; and (ii) a long-chain organic acid. As salts, the metals may be in a 2+, 3+, 4+, or 5+ oxidation state forming Metal(II), Metal(III), Metal(IV), and Metal(V) complexes with the long-chain organic acid. If an oxidation state is not specified the metal salt may comprise Metal(II), Metal(III), Metal(IV), and Metal(V) complexes.

The metal salts of long-chain organic acids may be M1 metal salts comprising the salt of an M1 metal and a long-chain organic acid. The metal salts of long-chain organic acids may be M2 metal salts comprising the salt of an M2 metal and a long-chain organic acid. The metal salts of long-chain organic acids may be M3 metal salts comprising the salt of an M3 metal and a long-chain organic acid. M1 metal salts, M2 metal salts, and M3 metal salts need not contain the same long-chain organic acid. Furthermore, M1 metal salts, M2 metal salts, and M3 metal salts may be formed in situ with a salt of a second organic acid and the M1, M2, or M3 metal element, and a long-chain organic acid.

In at least one embodiments, the M1 metal salt is selected from cobalt myristoleate, cobalt palmitoleate, cobalt cis-vaccenate, cobalt paullinate, cobalt oleate, cobalt gondoate, cobalt gadoleate, iron myristoleate, iron palmitoleate, iron cis-vaccenate, iron paullinate, iron oleate, iron gondoate, iron gadoleate, manganese myristoleate, manganese palmitoleate, manganese cis-vaccenate, manganese paullinate, manganese oleate, manganese gondoate, or manganese gadoleate.

In at least one embodiments, the M2 metal salt is selected from nickel myristoleate, nickel palmitoleate, nickel cis-vaccenate, nickel paullinate, nickel oleate, nickel gondoate, nickel gadoleate, zinc myristoleate, zinc palmitoleate, zinc cis-vaccenate, zinc paullinate, zinc oleate, zinc gondoate, zinc gadoleate, copper myristoleate, copper palmitoleate, copper cis-vaccenate, copper paullinate, copper oleate, copper gondoate, copper gadoleate, molybdenum myristoleate, molybdenum palmitoleate, molybdenum cis-vaccenate, molybdenum paullinate, molybdenum oleate, molybdenum gondoate, molybdenum gadoleate, tungsten myristoleate, tungsten palmitoleate, tungsten cis-vaccenate, tungsten paullinate, tungsten oleate, tungsten gondoate, tungsten gadoleate, silver myristoleate, silver palmitoleate, silver cis-vaccenate, silver paullinate, silver oleate, silver gondoate, or silver gadoleate.

In at least one embodiment, the M3 metal salt is selected from gallium myristoleate, gallium palmitoleate, gallium cis-vaccenate, gallium paullinate, gallium oleate, gallium gondoate, gallium gadoleate, indium myristoleate, indium palmitoleate, indium cis-vaccenate, indium paullinate, indium oleate, indium gondoate, indium gadoleate, scandium myristoleate, scandium palmitoleate, scandium cis-vaccenate, scandium paullinate, scandium oleate, scandium gondoate, scandium gadoleate, yttrium myristoleate, yttrium palmitoleate, yttrium cis-vaccenate, yttrium paullinate, yttrium oleate, yttrium gondoate, yttrium gadoleate, lanthanum myristoleate, lanthanum palmitoleate, lanthanum cis-vaccenate, lanthanum paullinate, lanthanum oleate, lanthanum gondoate, lanthanum gadoleate, cerium myristoleate, cerium palmitoleate, cerium cis-vaccenate, cerium paullinate, cerium oleate, cerium gondoate, cerium gadoleate, praseodymium myristoleate, praseodymium palmitoleate, praseodymium cis-vaccenate, praseodymium paullinate, praseodymium oleate, praseodymium gondoate, praseodymium gadoleate, neodymium myristoleate, neodymium palmitoleate, neodymium cis-vaccenate, neodymium paullinate, neodymium oleate, neodymium gondoate, neodymium gadoleate, gadolinium myristoleate, gadolinium palmitoleate, gadolinium cis-vaccenate, gadolinium paullinate, gadolinium oleate, gadolinium gondoate, gadolinium gadoleate, dysprosium myristoleate, dysprosium palmitoleate, dysprosium cis-vaccenate, dysprosium paullinate, dysprosium oleate, dysprosium gondoate, dysprosium gadoleate, holmium myristoleate, holmium palmitoleate, holmium cis-vaccenate, holmium paullinate, holmium oleate, holmium gondoate, holmium gadoleate, erbium myristoleate, erbium palmitoleate, erbium cis-vaccenate, erbium paullinate, erbium oleate, erbium gondoate, or erbium gadoleate.

The first dispersion system may also be formed by heating a mixture of a long-chain organic acid, a hydrocarbon solvent, and one or more metal salts of one or more second organic acids; and heating that mixture to T1. T1 may be a temperature at or higher than the lower of (i) the boiling point of the second organic acid or (ii) the decomposition temperature of the second organic acid. In some embodiments, the boiling point of the second organic acid is lower than T1. T1 may comprise temperatures from 50° C. to 350° C., such as 70° C. to 200° C., or 70° C. to 150° C. Heating at T1 may last from 10 min to 100 hours, such as from 10 min to 10 hours, 10 minutes to 5 hours, 10 minutes to 3 hours, or 10 minutes to 2 hours.

The second organic acid may comprise organic acids with a molecular weight lower than the molecular weight of the long-chain organic acids such as C8-organic acids, C1 to C7, C1 to C5, or C2 to C4 organic acids. Furthermore, the second organic acid may be more volatile than the long-chain organic acids. Some examples of suitable second acids are formic acid (bp 101° C.), acetic acid (bp 118° C.), propionic acid (bp 141° C.), butyric acid (bp 164° C.), lactic acid (bp 122° C.), citric acid (310° C.), ascorbic acid (decomp 190° C.), benzoic acid (249° C.), phenol (182° C.), acetylacetone (bp 140° C.), and acetoacetic acid (decomposition 80° C. to 90° C.). The second organic acid metal salts may comprise, for example, metal acetate, metal propionate, metal butyrate, metal lactate, metal acetylacetonate, or metal acetylacetate. Without being limited by theory, the second organic acid disposed on the metal may be released from the metal by exchange with the long-chain organic acid and the second organic acid may be removed under decreased pressure or flow of inert gas. The greater volatility of the second organic acid may allow for efficient exchange as the second organic acid is removed from solution. Removal of the second organic acid may also allow for formation of the first dispersion system in a single reaction vessel and may further allow for direct use in nanoparticle formation in the same reaction vessel.

In some embodiments, the long-chain organic solvent and the long-chain organic acid are mixed prior to addition of metals, sulfur, organosulfur, or organophosphorus forming a liquid pre-mixture. To the liquid pre-mixture may be added one or more metal salts of one or more second organic acids, and optionally elemental sulfur, organosulfur, organophosphorus, or combinations thereof.

The optional sulfur or organic sulfur compounds may comprise elemental sulfur, alkyl thiols, aromatic thiols, dialkyl thioethers, diaryl thioether, alkyl disulfides, aryldisulfides, or mixture(s) thereof, such as 1-dodecanethiol (bp 266° C. to 283° C.), 1-tridecanethiol (bp 291° C.), 1-tetradecanethiol (bp 310° C.), 1-pentadecanethiol (bp 325° C.), 1-hexadecanethiol (bp 343° C. to 352° C.), 1-heptadecanethiol (bp 348° C.), 1-octadecanethiol (bp 355° C. to 362° C.), 1-icosanethiol (mp bp 383° C.), 1-docosanethiol (bp 404° C.), 1-tetracosanethiol (bp 423° C.), decyl sulfide (bp 217° C. to 218° C.), dodecyl sulfide (bp 260° C. to 263° C.), thiophenol (bp 169° C.), diphenyl sulfide (bp 296° C.), diphenyl disulfide (bp 310° C.), or mixture(s) thereof. The sulfur or organic sulfur compounds may be soluble in the long-chain organic solvent. The amount of sulfur or organic sulfur included in the first dispersion system is set by the mole ratio to the metal(s) in the first dispersion system.

The optional organophosphorus compounds may comprise alkylphosphines, dialkyl phosphines, trialkylphosphines, alkylphosphineoxides, dialkyphosphineoxides, trialkylphosphineoxides, tetraalkylphosphonium salts, and mixtures thereof. For example, suitable organophosphorus compounds may include: tributylphosphine (bp 240° C.), tripentylphosphine (bp 310° C.), trihexylphosphine (bp 352° C.), diphneylphsophine (bp 280° C.), trioctylphosphine (bp 284° C. to 291° C.), triphenylphosphine (bp 377° C.), or mixture(s) thereof. The organic phosphorus compounds may be soluble in the long-chain organic solvent. The amount of organic phosphorus included in the first dispersion system is set by the mole ratio to the metal(s) in the first dispersion system.

The first dispersion system may be substantially free of surfactants other than salts of the long-chain organic acid.

The processes of producing nanoparticle compositions of this disclosure may comprise heating the first dispersion system to a second temperature (T2), where T2 is greater than T1 and no higher than the boiling point of the long-chain hydrocarbon solvent. T2 can promote at least a portion of the first dispersion system to decompose and form a second dispersion system comprising nanoparticles described in this disclosure dispersed in the long-chain hydrocarbon solvent.

The second temperature may comprise temperatures from T2a to T2b, where T2a and T2b can be, independently, e.g., 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450° C., as long as T2a<T2b. In some embodiments, T2a is 210° C.or greater, such as where T2a=210 and T1b=450; or where T1a=250 and T1b=350.

The M1 metal salt(s), M2 metal salt(s) (if any), and M3 metal salt(s) (if any) can decompose at the second temperature to form the kernels. The kernels may be solid particles comprising the metal and oxygen atoms. The long-chain organic acids or a portion thereof may partly remain attached to the kernel's surface. Without being limited by theory, oxygen atoms from the long-chain organic acids, may be included in the kernel as a portion of the surface oxygen atoms. Such partial attachments may be sufficient to withstand washing, centrifuging, and handling of the nanoparticles. Therefore, the nanoparticle composition may comprise kernels with long-chain hydrocarbyls attached to the surface of the kernels. Without being limited by theory, the long-chain hydrocarbyls attached to the kernel may allow for uniform dispersion in the second dispersion system and complete colloidal dissolution in hydrophobic solvents.

Furthermore, some portion of the long-chain organic acid salt may decompose to form an unsaturated compound (e.g. long-chain olefins) becoming a portion of the second dispersion system. The unsaturated compound may be identical to the long-chain hydrocarbon solvent if the solvent chosen is an alpha-olefin one carbon length shorter than the long-chain organic acid.

The decomposition of the metal salts forms kernels where two or three dimensions are from 4 nm to 100 nm in length, such as from 4 nm to 20 nm in length. The kernels can have a size distribution of 30% or less, 20% or less, 10% or less, or 5% or less, such as from 1% to 30%, from 5% to 20%, or from 5% to 10%. The size and size distribution are determined by TEM and SAXS.

The processes may take place in one or more reaction vessels under an inert atmosphere. The processes may comprise separating the nanoparticle composition from the long-chain hydrocarbon solvent. A suitable method of separating the nanoparticles from the long-chain hydrocarbon solvent may comprise addition of a counter-solvent causing precipitation of the nanoparticles. Suitable counter solvents may include: C1-C8 alcohols, such as C1-C6, C2-C4, or 1-butanol. Without being limited by theory, the increased polarity of the solution may cause the nanoparticles to precipitate out of solution where the counter solvent dissolves in the long-chain hydrocarbon solvent and long-chain organic acid mixture. Contaminants including unreacted metal salts, organic acids and corresponding salts may remain in the mixture of long-chain hydrocarbon solvent and counter-solvent and be removed in the process. The mixture of solvents and contaminants may be removed by centrifugation and decantation or filtration.

The processes may also include further purification of the nanoparticles by a cleaning process. The cleaning may comprise (i) dispersing the nanoparticles in a hydrophobic solvent such as benzene, pentane, toluene, hexanes, or xylenes; (ii) adding a counter solvent to precipitate the nanoparticles; and (iii) collecting the precipitate by centrifugation or filtration. Cleaning, comprising steps (i) through (iii), may be repeated to further purify the nanoparticles.

### Nanoparticle Support

Purified and/or unpurified nanoparticles may be dispersed in liquid media to form a nanoparticle dispersion. Suitable liquid media for forming a nanoparticle dispersion may include: benzene, pentane, toluene, hexanes, or xylenes. The nanoparticles may also be dispersed on a solid support by contacting the nanoparticle dispersion with the support. Suitable methods for contacting the nanoparticle dispersion with a solid support include: wet deposition, wet impregnation, or incipient wetness impregnation of the solid support. If the support is a large (greater than 100 nm) flat surface the nanoparticles may self-assemble into a monolayer on the support.

The supported nanoparticle composition of this disclosure comprises a support material (which may be called a carrier or a binder). The support material may be included at any suitable quantity, e.g., ≥20, ≥30, ≥40, ≥50, ≥60, ≥70, ≥80, ≥90, or even ≥95 wt %, based on the total weight of the supported nanoparticle composition. In supported nanoparticle compositions, the nanoparticle component can be suitably disposed on the internal or external surfaces of the support material. Support materials may comprise porous materials that provide mechanical strength and a high surface area. Non-limiting examples of suitable support materials can include: oxides (e.g. silica, alumina, titania, zirconia, or mixture(s) thereof), treated oxides (e.g. sulfated), crystalline microporous materials (e.g. zeolites), non-crystalline microporous materials, cationic clays or anionic clays (e.g. saponite, bentonite, kaoline, sepiolite, or hydrotalcite), carbonaceous materials, or combination(s) and mixture(s) thereof. A support material can be sometimes called a binder in a supported nanoparticle composition.

The supported nanoparticle composition of this disclosure may optionally comprise a solid diluent material. A solid diluent material is a solid material used to decrease nanoparticle to solid ratio and may be a material selected from conventional support materials. For example, a solid diluent material may be oxides (e.g. silica, alumina, titania, zirconia, or mixture(s) thereof), treated oxides (e.g. sulfated), crystalline microporous materials (e.g. zeolites), non-crystalline microporous materials, cationic clays or anionic clays (e.g. saponite, bentonite, kaoline, sepiolite, or hydrotalcite), carbonaceous materials, or combination(s) and mixture(s) thereof.

The nanoparticles can be combined with a support material, an optional promoter, or an optional solid diluent material, to form a supported nanoparticle composition. The combination of the support material and the nanoparticles can be processed in any suitable forming processes, including but not limited to: grinding, milling, sifting, washing, drying, calcination, and the like. Drying or calcining the nanoparticles, optional promoter, and optional solid diluent material, on a support produces a catalyst composition. Drying and Calcining may take place at a third temperature (T3). The third temperature may comprise temperatures from T3a to T3b, where T3a and T3b can be, independently, e.g., 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650° C., as long as T2a<T2b. In some embodiments, T2a is 500° C.or greater, such as where T2a=500° C. and T1b=650° C.; or where T1a=550° C. and T1b=600° C. The catalyst composition may be then disposed in a conversion reactor to perform its intended function, such as a syngas converting reactor in a syngas converting process.

It is also contemplated that the nanoparticles may be combined or formed with a precursor of a support material to obtain a catalyst composition precursor mixture. Suitable precursors of various support materials can include, e.g., alkali metal aluminates, water glass, a mixture of alkali metal aluminates and water glass, a mixture of sources of a di-, tri-, and/or tetravalent metal, such as a mixture of water-soluble salts of magnesium, aluminum, and/or silicon, chlorohydrol, aluminum sulfate, or mixture(s) thereof. The catalyst composition precursor mixture comprising the support and nanoparticles is subsequently subject to drying and calcining, resulting in the formation of the catalyst composition and the support material substantially in the same step.

A promoter may be added to a supported nanoparticle composition or a catalyst composition forming a catalyst precursor composition. The catalyst precursor may be dried and/or calcined to form a catalyst composition comprising a promoter. Promoters may include sulfur, phosphorus, or salts of elements selected from Groups 1, 7, 11, or 12 of the periodic table, such as Li, Na, K, Rb, Cs, Re, Cu, Zn, Ag, and mixture(s) thereof. Typically, sulfide and sulfate salts are used. For example, a promoter may be added to a supported nanoparticle composition or a catalyst composition as part of a solution, the solvent can then be removed via evaporation (e.g. an aqueous solution where the water is later removed).

Without being bound by a particular theory, it is believed that the metal oxide(s), and possibly the elemental phases of M1 in the kernel provide the catalytic activity for chemical conversion processes such as a Fischer-Tropsch synthesis. One or more of M2 and/or M3 can provide direct catalytic function as well. In addition, one or more of M2 and/or M3 can perform the function of a “promoter” in the supported nanoparticle composition. Furthermore, sulfur and or phosphorus, if present, can perform the function of a promoter in the catalyst composition as well. Promoters typically improve one or more performance properties of a catalyst. Example properties of catalytic performance enhanced by inclusion of a promoter in a catalyst over the catalyst composition without a promoter, may include: selectivity, activity, stability, lifetime, regenerability, reducibility, and resistance to potential poisoning by impurities such as sulfur, nitrogen, and oxygen.

It may be advantageous for the nanoparticles to be uniformly dispersed on the support. The nanoparticles can be substantially homogeneously distributed in the supported nanoparticle composition, resulting in a highly dispersed distribution, which can contribute to a high catalytic activity of a catalyst composition.

The synthesis methods disclosed may produce crystalline kernels with uniform particle shape and size. The kernels comprise metal oxide(s) that may be uniformly distributed throughout the kernel, which may improve catalysis when the kernel is included in a catalyst composition. The kernel may be part of a nanoparticle which may comprise long-chain hydrocarbons disposed on the kernel. The nanoparticles may be formed in a single reaction vessel from readily available precursors. The nanoparticle may be dispersed in liquid media, and thereby dispersed on a solid support. The nanoparticles dispersed on solid support may together be dried and or calcined to form a catalyst composition

### II.2 Processes for Converting Syngas for Making LAOs

The catalyst composition of the second aspect of this disclosure can be advantageously used as a Fischer-Tropsch catalyst in a process for making LAOs from a feed comprising syngas.

The term “syngas” as used herein relates to a gaseous mixture consisting essentially of hydrogen (H₂) and carbon monoxide (CO). The syngas, which is used as a feed stream, may include up to 10 mol % of other components such as CO₂ and lower hydrocarbons (lower HC), depending on the source and the intended conversion processes. Said other components may be side-products or unconverted products obtained in the process used for producing the syngas. The syngas may contain such a low amount of molecular oxygen (O₂) so that the quantity of O₂ present does not interfere with the Fischer-Tropsch synthesis reactions and/or other conversion reactions. For example, the syngas may include not more than 1 mol % O₂, not more than 0.5 mol % O₂, or not more than 0.4 mol % O₂. The syngas may have a hydrogen (H₂) to carbon monoxide (CO) molar ratio of from r1 to r2, where r1 and r2 can be, independently, e.g., 0.3, 0.4, 0.5, 0.6, 0.7, 0.8, 0.9, 1, 1.2, 1.4, 1.5, 1.6, 1.8, 2.0, 2.2, 2.4, 2.5, 2.6, 2.8, 3.0, as long as r1<r2. The partial pressures of H₂ and CO may be adjusted by introduction of inert gas to the reaction mixture.

Syngas can be formed by reacting steam and/or oxygen with a carbonaceous material, for example, natural gas, coal, biomass, or a hydrocarbon feedstock through a reforming process in a syngas reformer. The reforming process can be based on any suitable reforming process, such as Steam Methane Reforming, Auto Thermal Reforming, or Partial Oxidation, Adiabatic Pre Reforming, or Gas Heated Reforming, or a combination thereof. Example steam and oxygen reforming processes are detailed in U.S. Pat. No. 7,485,767.

The syngas formed from steam or oxygen reforming includes hydrogen and one or more carbon oxides (CO and CO₂). The hydrogen to carbon oxide ratio of the syngas produced will vary depending on the reforming conditions used. The syngas reformer product(s) should contain H₂, CO and CO₂ in amounts and ratios which render the resulting syngas blend suitable for subsequent processing into either oxygenates comprising methanol/dimethyl ether or in Fischer-Tropsch synthesis.

It is possible to alter the ratio of components within the syngas and the absolute CO₂ content of the syngas by removing, and optionally recycling, some of the CO₂ from the syngas produced in one or more reforming processes. Several commercial technologies are available (e.g. acid gas removal towers) to recover and recycle CO₂ from syngas as produced in the reforming process. In at least one embodiment, CO₂ can be recovered from the syngas effluent from a steam reforming unit, and the recovered CO₂ can be recycled to a syngas reformer.

Suitable Fischer-Tropsch catalysis procedures may be found in: U.S. Pat. Nos. 7,485,767; 6,211,255; and 6,476,085; the relevant portions of their contents being incorporated herein by reference. The catalyst composition may be contained in a fixed bed reactor, a fluidized bed reactor, or any other suitable reactor.

The conversion conditions may include a wide range of temperatures. In at least one embodiment, the conversion conditions comprise a temperature in a range from T1 to T2° C., where T1 and T2 can be., e.g., 175, 180, 190, 200, 220, 240, 250, 260, 280, 290, 300, 320, 340, 350, as long as T1<T2.

The conversion conditions may include a wide range of pressures. In at least one embodiment, the absolute reaction pressure ranges from p1 to p2 kilopascal (“kPa”), wherein p1 and p2 can be, independently, e.g., 100, 150, 200, 250, 300, 350, 400, 450, 500, 600, 700, 800, 900, 1000, 1500, 2000, 2500, 3000, as long as p1<p2.

The conversion conditions may comprise a gas hourly space velocity from v1 to v2 hr-1, wherein v1 and v2 can be, independently, e.g., 500, 600, 700, 800, 900, 1000, 2000, 3000, 4000. 5000, 6000, 7000, 8000, 9000, 10000, as long as v1<v2.

In certain embodiments, the catalyst composition may be activated in the presence of a H2-containing atmosphere before step (I). Such activation can be performed ex-situ outside of the conversion reactor before being loaded into the conversion reactor, or alternatively or additionally, in-situ inside the conversion reactor. Such H2-containing atmosphere can be, e.g., syngas, high-purity hydrogen, H2/inert gas mixture, and mixtures thereof. Inert gas can be, e.g., N₂, He, Ne, Ar, and Kr. The activation may be performed at a temperature from, e.g., T1 to T2° C., where T1 and T2 can be, independently, e.g., 150, 160, 170, 180, 190, 200, 220, 240, 250, 260, 280, 300, 320, 340, 350, 360, 380, 400, 420, 440, 450, 460, 480, 500, as long as T1<T2.

In certain embodiments of the process of the second aspect of this disclosure, in step (I), the conversion product mixture comprises LAOs, in aggregate, from c1 to c2 mol %, based on the total moles of the reaction product mixture, where c1 and c2 can be, independently, e.g., 40, 45, 50, 55, 60, 65, 70, 75, 80, 85, 90, 91, 92, 93, 94, 95, as long as c1<c2. Such LAOs can advantageously comprise 1-butene, 1-pentene, 1-hexene, 1-heptene, and 1-octene.

In certain embodiments of the process of the second aspect of this disclosure, in step (I), at least one of the following is met:

(i) the conversion product mixture comprises 1-butene from c41 to c42 mol %, based on the total moles of the C4 compounds in the conversion product mixture, where c41 and c42 can be, independently, e.g., 40, 45, 50, 55, 60, 65, 70, 75, 80, 85, 90, 91, 92, 93, 94, 95, as long as c41<c42. Preferably c41=60. Preferably c41=80;
(ii) the conversion product mixture comprises 1-pentene from c51 to c52 mol %, based on the total moles of the C5 compounds in the conversion product mixture, where c51 and c52 can be, independently, e.g., 40, 45, 50, 55, 60, 65, 70, 75, 80, 85, 90, 91, 92, 93, 94, 95, as long as c51<c52. Preferably c51=50. Preferably c51=70;
(iii) the conversion product mixture comprises 1-hexene from c61 to C62 mol %, based on the total moles of the C6 compounds in the conversion product mixture, where c61 and c62 can be, independently, e.g., 40, 45, 50, 55, 60, 65, 70, 75, 80, 85, 90, 91, 92, 93, 94, 95, as long as c61<c62. Preferably c61=50. Preferably c61=70;
(iv) the conversion product mixture comprises 1-heptene from c71 to c72 mol %, based on the total moles of the C7 compounds in the conversion product mixture, where c71 and c72 can be, independently, e.g., 40, 45, 50, 55, 60, 65, 70, 75, 80, 85, 90, 91, 92, 93, 94, 95, as long as c71<c72. Preferably c71=50. Preferably c51-60; and

(v) the conversion product mixture comprises 1-octene from c81 to c82 mol %, based on the total moles of the C8 compounds in the conversion product mixture, where c81 and c82 can be, independently, e.g., 40, 45, 50, 55, 60, 65, 70, 75, 80, 85, 90, 91, 92, 93, 94, 95, as long as c81<c82. Preferably c81=50. Preferably c81=60.

In certain embodiments of the process of the second aspect of this disclosure, in step (I), at least one of the following is met:

(i) the conversion product mixture comprises n-butane from c4-1 to c4-2 mol %, based on the total moles of the C4 compounds in the conversion product mixture, where c4-1 and c4-2 can be, independently, e.g., 5, 10, 15, 20, 25, 30, 35, 40, 45, 50, as long as c4-1<c4-2. Preferably c4-2-40. Preferably c4-2=30;

(ii) the conversion product mixture comprises n-pentane from c5-1 to c5-2 mol %, based on the total moles of the C5 compounds in the conversion product mixture, where c5-1 and c5-2 can be, independently, e.g., 5, 10, 15, 20, 25, 30, 35, 40, 45, 50, as long as c5-1<c5-2. Preferably c5-2=40. Preferably c5-2=30;

(iii) the conversion product mixture comprises n-hexane from c6-1 to C6-2 mol %, based on the total moles of the C6 compounds in the conversion product mixture, where c6-1 and c6-2 can be, independently, e.g., 5, 10, 15, 20, 25, 30, 35, 40, 45, 50, as long as c6-1<c6-2. Preferably c6-2=40. Preferably c6-2=30;

(iv) the conversion product mixture comprises n-heptane from c7-1 to c7-2 mol %, based on the total moles of the C7 compounds in the conversion product mixture, where c7-1 and c7-2 can be, independently, e.g., 5, 10, 15, 20, 25, 30, 35, 40, 45, 50, as long as c7-1<c7-2. Preferably c7-2=40. Preferably c7-2=30; and

(v) the conversion product mixture comprises n-octane from c8-1 to c8-2 mol %, based on the total moles of the C8 compounds in the conversion product mixture, where c8-1 and c8-2 can be, independently, e.g., 5, 10, 15, 20, 25, 30, 35, 40, 45, 50, as long as c8-1<c8-2. Preferably c8-2=40. Preferably c8-2=30.

In certain embodiments of the process of the second aspect of this disclosure, in step (I), at least one of the following is met, resulting in the production of internal olefins at low concentrations in the conversion product mixture:

(i) the conversion product mixture comprises 2-butene from c4-a to c4-b mol %, based on the total moles of the C4 compounds in the conversion product mixture, where c4-a and c4-2 can be, independently, e.g., 0, 2, 4, 5, 6, 8, 10, 12, 14, 15, 16, 18, 20, as long as c4-a<c4-b. Preferably c4-b=15. Preferably c4-5=10;

(ii) the conversion product mixture comprises C5 internal olefins from c5-a to c5-b mol %, based on the total moles of the C5 compounds in the conversion product mixture, where c5-a and c5-2 can be, independently, e.g., 0, 2, 4, 5, 6, 8, 10, 12, 14, 15, 16, 18, 20, as long as c5-a<c5-b. Preferably c5-b=15. Preferably c5-b=10;

(iii) the conversion product mixture comprises C6 internal olefins from c6-a to C6-b mol %, based on the total moles of the C6 compounds in the conversion product mixture, where c6-a and c6-2 can be, independently, e.g., 0, 2, 4, 5, 6, 8, 10, 12, 14, 15, 16, 18, 20, as long as c6-a<c6-b. Preferably c6-b=15. Preferably c6-b=10;

(iv) the conversion product mixture comprises C7 internal olefins from c7-a to c7-b mol %, based on the total moles of the C7 compounds in the conversion product mixture, where c7-a and c7-2 can be, independently, e.g., 0, 2, 4, 5, 6, 8, 10, 12, 14, 15, 16, 18, 20, as long as c7-a<c7-b. Preferably c7-b=15. Preferably c5-b=10; and

(v) the conversion product mixture comprises C8 internal olefins from c8-a to c8-b

mol %, based on the total moles of the C8 compounds in the conversion product mixture, where c8-a and c8-2 can be, independently, e.g., 0, 2, 4, 5, 6, 8, 10, 12, 14, 15, 16, 18, 20, as long as c8-a<c8-b. Preferably c8-b=15. Preferably c8-b=10.

In certain embodiments of the process of the second aspect of this disclosure, in step (I), at least one of the following is met, resulting in the production of linear 1-alchols at low concentrations in the conversion product mixture:

(i) the conversion product mixture comprises 1-butanol from c4-m to c4-n mol %, based on the total moles of the C4 compounds in the conversion product mixture, where c4-m and c4-2 can be, independently, e.g., 0, 0.1, 0.2, 0.2, 0.3, 0.4, 0.5, 0.6, 0.7, 0.8, 0.9, 1, 1.5, 2, 2.5, 3, 3.5, 4, 4.5, 5, as long as c4-m<c4-n. Preferably c4-n=2. Preferably c4-2=1; more preferably c4-n=0;

(ii) the conversion product mixture comprises 1-pentanol from c5-m to c5-n mol %, based on the total moles of the C5 compounds in the conversion product mixture, where c5-m and c5-2 can be, independently, e.g., 0, 0.1, 0.2, 0.2, 0.3, 0.4, 0.5, 0.6, 0.7, 0.8, 0.9, 1, 1.5, 2, 2.5, 3, 3.5, 4, 4.5, 5, as long as c5-m<c5-n. Preferably c5-n=2. Preferably c5-2=1; more preferably c5-n=0;

(iii) the conversion product mixture comprises 1-hexanol from c6-m to C6-n mol %, based on the total moles of the C6 compounds in the conversion product mixture, where c6-m and c6-2 can be, independently, e.g., 0, 0.1, 0.2, 0.2, 0.3, 0.4, 0.5, 0.6, 0.7, 0.8, 0.9, 1, 1.5, 2, 2.5, 3, 3.5, 4, 4.5, 5, as long as c6-m<c6-n. Preferably c6-n=2. Preferably c6-2=1; more preferably c6-n=0;

(iv) the conversion product mixture comprises 1-heptanol from c7-m to c7-n mol %, based on the total moles of the C7 compounds in the conversion product mixture, where c7-m and c7-2 can be, independently, e.g., 0, 0.1, 0.2, 0.2, 0.3, 0.4, 0.5, 0.6, 0.7, 0.8, 0.9, 1, 1.5, 2, 2.5, 3, 3.5, 4, 4.5, 5, as long as c7-m<c7-n. Preferably c7-n=2. Preferably c7-2=1; more preferably c7-n=0; and

(v) the conversion product mixture comprises 1-octanol from c8-m to c8-n mol %, based on the total moles of the C8 compounds in the conversion product mixture, where c8-m and c8-2 can be, independently, e.g., 0, 0.1, 0.2, 0.2, 0.3, 0.4, 0.5, 0.6, 0.7, 0.8, 0.9, 1, 1.5, 2, 2.5, 3, 3.5, 4, 4.5, 5, as long as c8-m<c8-n. Preferably c8-n=2. Preferably c8-2=1; more preferably c8-n=0.

In certain embodiments, the conversion product mixture is substantially free of alcohols.

One or more LAO products can be produced by separating the conversion product mixture. Any suitable separating processes and equipment maybe used, including but not limited to flashing, distillation, membrane separation, absorption, adsorption, cryogenic separation, crystallization, and the like.

### II-3. EXAMPLES

### Example II-3-1. Preparation of MnCoO, Spherical Nanoparticles

A reaction solution was prepared by dissolving manganese (II) acetylacetonate (Mn(CH₃COCHCOCH₂)₂) and Cobalt (II) acetate tetrahydrate (Co(CH₃COO)₂·4H₂O) in a mixture of oleic acid (OLAC) and 1-octadecene. The reaction solution had a molar ratio of 4.5 mol OLAC:mol metal and a combined metal concentration of 0.164 mmol Mn/mL of 1-octadecene. The reaction solution was heated to a temperature of 130° C. under flow of nitrogen and held at 130° C. for 90 minutes. The mixture was then heated under an inert atmosphere of nitrogen at a rate of 10° C./min to reflux (320° C.) The reaction mixture was held at 320° C.for 30 min. The reaction mixture was cooled under an inert atmosphere using a flow of RT air to cool the exterior of the reaction vessel. The nanoparticles were collected and purified via repeated washing and decanting/centrifugation steps using hexane as a hydrophobic solvent, and isopropanol as a counter solvent. The purified nanoparticles were dispersed in toluene. TEM imagery shows that the nanoparticles are roughly spherical in shape, have an average diameter of 6.3 nanometers and a size distribution of 13%.

### Example II-3-1a. Supporting MnCoOₓ Spherical Nanoparticles with Silica

Eight grams of SiO₂ was dispersed in hexane. While under vigorous stirring, the nanoparticle solution prepared according to Example II-3-1 was added to the support dispersion and the mixture was stirred at 25° C. for 180 minutes. The catalyst powder was recovered by centrifugation. The powder was washed three times with hexane via sonication and centrifugation. The supported nanoparticle composition was dried at 60° C. for 12 hours under vacuum (100 mmHg). The dried supported nanoparticle powder was then calcined in static air at 325° C. for 180 minutes using heating and cooling ramps of 3° C. per minute.

### Example II-3-1b. Supporting MnCoOₓ Spherical Nanoparticles with Alumina

Eight grams of Al₂O₃ was dispersed in hexane. While under vigorous stirring, the nanoparticle solution prepared according to Example 1 was added to the support dispersion and the mixture was stirred at 25° C. for 180 minutes. The catalyst powder was recovered by centrifugation. The powder was washed three times with hexane via sonication and centrifugation. The supported nanoparticle composition was dried at 60° C. for 12 hours under vacuum (100 mmHg). The dried supported nanoparticle powder was then calcined in static air at 325° C. for 180 minutes using heating and cooling ramps of 3° C. per minute.

### Example II-3-2. Preparation of Supported MnCoOₓ Rod-Shaped Nanoparticles

A reaction solution was prepared by dissolving manganese (II) acetylacetonate acetate (Mn(CH₃COCHCOCH₂)₂) and Cobalt (II) acetate tetrahydrate (Co(CH₃COO)₂·4H₂O) in a mixture of oleic acid (OLAC) and 1-octadecene. The reaction solution had a molar ratio of 4.5 mol OLAC: mol Metal (Mn+Co) and a combined metal concentration of 0.9 mmol Mn/mL of 1-octadecene. The reaction solution was heated to a temperature of 130° C. under flowing nitrogen and held at 130° C. for 60 minutes. The mixture was then heated under an inert atmosphere of nitrogen at a rate of 10° C./min to reflux (320° C.). The reaction mixture was held at 320° C. for 120 min. The reaction mixture was cooled under an inert atmosphere using a flow of RT air to cool the exterior of the reaction vessel. The nanoparticles were collected and purified via repeated washing and decanting/centrifugation steps using hexane as a hydrophobic solvent, and isopropanol as a counter solvent. The purified nanoparticles were dispersed in toluene. TEM images illustrated that the nanoparticles are rod-shaped, have an average length of 64.1 with a length distribution of 15% and an average width of 11.7 nanometers with a width distribution of 13%.

### Comparative Example II-3-3. Bulk Co₂MnO₄

An aqueous solution was prepared containing 48.9 g of Co(NO₃)₂-6H₂O and 24.1 g of Mn(NO₃)-2.6H₂O in 125 ml water. This was added to an aqueous solution of 48.4 g citric acid and 14.1 mi ethylene glycol in 25 ml water with stirring at 70° C. to 90° C. The mixture became thick after 1-2 hours, after which it was calcined in air at 350° C. for 30 minutes.

### Example II-3-4. Process for Converting Syngas Using Supported MnCoOₓ Spherical Nanoparticles on Alumina for Making LAOs

A 1:3 mixture of MnCoOₓ(spherical)/Al₂O₃ of Example II-3-1b and silicon carbide (both components sized between 40-60 mesh) were loaded into a fixed-bed reactor system. The mixture was dried at 110° C. under an N₂ purge at 15 bar pressure and GHSV=2000 h⁻¹ for 2 h. The feed gas was switched to a 2:1 CO:H₂ mixture, the reactor pressure at 15 bar, and GHSV=2000 h⁻¹, and the reactor temperature increased to 270° C. at a 1° C./min ramp rate. The reactor was kept at 270° C. for 2 h and the reactor was cooled to the Fischer-Tropsch synthesis temperature (e.g., 250° C.). Once the temperature stabilized, the reactor feed was switched to a mixture of H₂ and CO. The range of conditions explored were as follows: (1) temperature, 150-350° C.; (2) pressure, 1-50 bar; (3) H₂:CO ratio, 1:3 to 3:1, and (4) GHSV, 1000-10,000 h⁻¹. The product mixture from the reactor was analyzed for composition. Selectivities in the product mixture are reported in TABLE VII below.

TABLE VII

| Product | Product Type | Carbon Number | Selectivity (%) |
|---|---|---|---|
| 1-Butene | LAO | 4 | 4.9 |
| n-Butane | Paraffin | 4 | 1.3 |
| 1-Pentene | LAO | 5 | 5.0 |
| n-Pentane | Paraffin | 5 | 1.3 |
| 1-Hexanol | Alcohol | 6 | 0.1 |
| 1-Hexene | LAO | 6 | 4.8 |
| n-Hexane | Paraffin | 6 | 1.2 |
| 1-Heptene | LAO | 7 | 4.2 |
| trans-2-Heptene | LIO | 7 | 0.1 |
| n-Heptane | Paraffin | 7 | 1.5 |
| 1-Octene | LAO | 8 | 3.6 |
| cis-2-Octene | LIO | 8 | 0.2 |
| trans-2-Octene | LIO | 8 | 0.1 |
| n-Octane | Paraffin | 8 | 1.6 |

As can be clearly seen from TABLE VII above, the MnCoOx(spherical)/Al₂O₃ containing catalyst demonstrated very high selectivities for C4, C5, C6, C7, and C8 LAOs in the product mixture, which are all much higher than (i) those for C4, C5, C6, C7, and C8 n-paraffins; and (ii) those for C4, C5, C6, C7, and C8 LIOs, respectively. Surprisingly, negligible to no linear C4, C5, C6, C7, and C8 primary alcohols were produced. The MnCoOx nanoparticles/Al₂O₃ catalyst proved to be a surprisingly advantageous catalyst for converting syngas to LAOs.

This disclosure can further include one or more of the following aspects/embodiments:

A1. A process for making linear alpha-olefin(s), the process comprising:

(I) contacting a feed comprising syngas with a catalyst composition in a conversion reactor under conversion conditions to produce a conversion product mixture comprising a linear alpha-olefin, wherein the catalyst composition comprises:
a support; and
a plurality of nanoparticles on the support, wherein:
each nanoparticle comprises a kernel, the kernels have an average particle size from 4 to 100 nm and a particle size distribution of no greater than 20%; the kernels comprise oxygen, a metal element M1, optionally sulfur, optionally phosphorus, an optional metal element M2, and optionally a third metal element M3, where:
M1 is selected from Mn, Fe, Co, and combination of two or more thereof;
M2 is selected from Ni, Zn, Cu, Mo, W, Ag, and combinations thereof;
M3 is selected from Y, Sc, alkaline metals, the lanthanides, group 13, 14, or 15 elements, and combinations thereof; and
the molar ratios of M2, M3, S, and P, if any, to M1 is r1, r2, r3, and r4, respectively, 0≤r1≤2, 0≤r2≤2, 0≤r3≤5, and 0≤r4≤5; and

(II) obtaining a linear alpha-olefin product from the conversion product mixture.

A2. The process of A1, wherein 0.05≤r1≤0.5, and 0.005≤r2≤0.5.

A3. The process of A1 or A2, wherein the kernels comprise an oxide of the at least one metal element selected from M1, M2, and M3.

A4. The process of any of A1 to A3, wherein the nanoparticles have an average particle size of from 4 to 20 nm.

A5. The process of any of A1 to A4, wherein the nanoparticles have a particle size distribution of from 5 to 15%.

A6. The process of any of A1 to A5, wherein the kernels comprise at least two metal elements.

A7. The process of any of A1 to A6, wherein the nanoparticles are substantially free of long-chain groups attached to the surface of the kernels.

A8. The process of A1 to A7, wherein the kernels consist essentially of oxygen, M1, optionally M2, optionally M3, optionally sulfur, and optionally phosphorus.

A9. The process of any of A1 to A8, wherein the kernels are substantially spherical.

A10. The process of any of A1 to A8, wherein the kernels are rod-shaped.

A11. The process of any of A1 to A10, wherein at least one of the following is met:

(i) the feed has a H₂ to CO molar ratio ranging from 0.3 to 3.0, preferably 0.5 to 2.5;

(ii) in step (I), the conversion conditions comprises at least one of the following:

a temperature in a range from 175 to 350° C., preferably 180 to 300° C.;

a pressure in a range from 100 to 3,000 kilopascal absolute, preferably 100 to 2,000 kilopascal absolute; and

a GHSV ranging from 500 to 10,000 hour-1, preferably 1,000 to 5,000 hour-1.

A12. The process of any of A1 to A11, wherein the process further comprises, before step (I):

(I0) activating the catalyst composition in the presence of a H2-containing atmosphere.

A13. The process of A12, wherein the H2-containing atmosphere is selected from syngas, hydrogen, a mixture of H2 and an inert gas, and mixtures thereof.

A14. The process of A12 or A13, wherein step (10) is performed at a temperature in a range from 150 to 500° C.

A15. The process of any of A1 to A14, wherein in step (I), the conversion product mixture comprises linear alpha-olefins, in aggregate, from 40 to 95 mol %, based on the total moles of the reaction product mixture.

A16. The process of any of A1 to A15, wherein in step (I), at least one of the following is met:

(i) the conversion product mixture comprises 1-butene from 40 to 95 mol %, based on the total moles of the C4 compounds in the conversion product mixture;

(ii) the conversion product mixture comprises 1-pentene from 40 to 95 mol %, based on the total moles of the C5 compounds in the conversion product mixture;

(iii) the conversion product mixture comprises 1-hexene from 40 to 95 mol %, based on the total moles of the C6 compounds in the conversion product mixture;

(iv) the conversion product mixture comprises 1-heptene from 40 to 95 mol %, based on the total moles of the C7 compounds in the conversion product mixture;

and

(v) the conversion product mixture comprises 1-octene from 40 to 95 mol %, based on the total moles of the C8 compounds in the conversion product mixture.

A17. The process of any of A1 to A16, wherein in step (I), at least one of the following is met:

(i) the conversion product mixture comprises n-butane from 5 to 50 mol %, based on the total moles of the C4 compounds in the conversion product mixture;

(ii) the conversion product mixture comprises n-pentane from 5 to 50 mol %, based on the total moles of the C5 compounds in the conversion product mixture;

(iii) the conversion product mixture comprises n-hexane from 5 to 50 mol %, based on the total moles of the C6 compounds in the conversion product mixture;

(iv) the conversion product mixture comprises n-heptane from 5 to 50 mol %, based on the total moles of the C7 compounds in the conversion product mixture;

and

(v) the conversion product mixture comprises n-octane from 5 to 50 mol %, based on the total moles of the C8 compounds in the conversion product mixture.

A18. The process of any of A1 to A17, wherein in step (I), at least one of the following is met:

(i) the conversion product mixture comprises 2-butene from 0 to 20 mol %, based on the total moles of the C4 compounds in the conversion product mixture;

(ii) the conversion product mixture comprises C5 internal olefins from 0 to 20 mol %, based on the total moles of the C5 compounds in the conversion product mixture;

(iii) the conversion product mixture comprises C6 internal olefins from 0 to 20 mol %, based on the total moles of the C6 compounds in the conversion product mixture;

(iv) the conversion product mixture comprises C7 internal olefins from 0 to 20 mol %, based on the total moles of the C7 compounds in the conversion product mixture;

and

(v) the conversion product mixture comprises C8 internal olefins from 0 to 20 mol %, based on the total moles of the C8 compounds in the conversion product mixture.

A19. The process of any of A1 to A18, wherein the conversion produce mixture is substantially free of linear 1-alcohols.

A20. The process of A19, wherein the conversion product mixture is substantially free of alcohols.

A21. The process of any of A1 to A20, wherein the catalyst composition is made by a catalyst fabrication process comprising:

(A) providing a nanoparticle dispersion comprising a liquid medium and a plurality of nanoparticles distributed therein, wherein each nanoparticle comprises a kernel, the kernels have an average particle size from 4 to 100 nm and a particle size distribution of 20% or less; the kernels comprise oxygen, a metal element M1, optionally sulfur, optionally phosphorus, optionally a second metal element M2, and optionally a third metal element M3, where:
M1 is selected from Mn, Fe, Co, or a combination of two or more thereof;
M2 is selected from Ni, Zn, Cu, Mo, W, Ag, and combinations thereof;
M3 is selected from Y, Sc, alkaline metals, the lanthanides, group 13, 14, and 15 elements, and combinations thereof; and
the molar ratios of M2, M3, S, and P, if any, to M1 is r1, r2, r3, and r4, respectively, 0≤r1≤2, 0≤r2≤2, 0≤r3<5, and 0≤r4≤5; and

(B) disposing a quantity of the nanoparticle dispersion on a support to obtain a supported nanoparticle composition.

A22. The process of A21, wherein the catalyst fabrication process further comprises:

(C) calcining the supported nanoparticle composition to obtain a catalyst composition.

A23. The process of A21 or A22, wherein the catalyst fabrication process further comprises:

(D) impregnating the support, the supported nanoparticle composition, or catalyst composition with a precursor of a promoter to obtain a catalyst precursor; and

(E) drying and/or calcining the catalyst precursor to obtain a catalyst composition comprising a promoter.

A24. The process of any of A21 to A23, wherein step (A) comprises:

(A1) providing a first dispersion system at a first temperature, the first dispersion system comprising a salt of a long-chain organic acid and M1, optionally a salt of the long-chain organic acid and M2 optionally a salt of the long-chain organic acid and M3, a long-chain hydrocarbon solvent, optionally a salt of a second organic acid and M1, optionally a salt of a third organic acid and M2, optionally a salt of a fourth organic acid and M3, optionally sulfur or an organic sulfur compound soluble in the long-chain hydrocarbon solvent, and optionally an organic phosphorous compound soluble in the long-chain hydrocarbon solvent; and

(A2) heating the first dispersion system to a second temperature higher than the first temperature but no higher than the boiling point of the long-chain hydrocarbon solvent, where at least a portion of the salt(s) of the long-chain organic acid and at least a portion of the salt(s) of the second organic acid, if present, to form a second dispersion system comprising nanoparticles dispersed in the long-chain hydrocarbon solvent, and the nanoparticles comprise kernels, and the kernels comprise M1, optionally M2, optionally M3, oxygen, optionally sulfur, and optionally phosphorus;

(A3) separating the nanoparticles from the second dispersion system; and

(A4) dispersing the nanoparticles separated in (A3) in the liquid medium to form the nanoparticle dispersion.

A25. The process of any of A21 to A24, wherein the nanoparticles have an average particle size in a range from 4 to 20 nm, and a particle size distribution of no greater than 20%.

A26. The process of A24 or A25, wherein steps (A1), (A2), (A3), and (A4) are performed in the same vessel.

A27. The process of any of A21 to A26, wherein the second organic acid has a boiling point lower than the first temperature.

A28. The process of any of A21 to A22, wherein the first dispersion system is substantially free of a surfactant other than the salt(s) of the long-chain organic acid.

A29. The process of any of A21 to A28, wherein the second temperature is in a range

from 210° C. to 450° C.

A30. The process of any of A21 to A29, wherein the long-chain organic acid and the long-chain hydrocarbon solvent do not differ in number of average carbon atoms per molecule by more than 4.

B1. A process for making linear alpha-olefin(s), the process comprising:

(I) contacting a feed comprising syngas with a catalyst composition in a conversion reactor under conversion conditions to produce a conversion product mixture comprising a linear alpha-olefin, wherein the catalyst composition comprises a catalytic component, wherein the catalytic component comprises:

a metal element M¹, selected from iron, cobalt, manganese, and combinations of two or more thereof at any proportion;

a metal element M², selected from aluminum, gallium, indium, thallium, chromium, scandium, yttrium, the lanthanide series, the actinide series, and any combination of two or more thereof at any proportion;

an optional metal M³, differing from M¹ and M²;

carbon;

nitrogen; and

optionally sulfur, at a molar ratio of M², M³, carbon, nitrogen, and sulfur to M¹ of r1, r2, r3, r4, and r5, respectively, indicated below:

M²:M³:C:N:S:M¹=r1:r2:r3:r4:r5:1, where:

0.1≤r1≤1.5;

0≤r2≤0.5;

0<r3≤1;

0<r4≤1; and

0≤r5≤1; and

(II) obtaining a linear alpha-olefin product from the conversion product mixture.

B2. The process of B1, wherein at least a portion of the carbon in the catalytic component is present as a metal carbide of one or more of M¹, M², and M³, and at least a portion of the nitrogen in the catalytic component is present as a metal nitride of one or more of M¹, M², and M³, as determined by x-ray diffraction diagram of the catalytic component.

B3. The process of B2, wherein the metal carbide and/or the metal nitride are distributed homogenously in the catalytic component.

B4. The process of any of B1 to B3, wherein M¹ is selected from iron, cobalt, combinations of iron and cobalt at any proportion, combinations of iron and manganese at any proportion, combination of cobalt with manganese at any proportion, and combination of iron, cobalt, and manganese at any proportion.

B5. The process of any of B1 to B4, wherein M² is selected from yttrium and the lanthanide series.

B6. The process of any of B1 to B5, wherein M³ is selected from alkali metals, copper, silver, and any combinations and mixtures of two or more thereof at any proportion.

B7. The process of any of B1 to B6, wherein the catalytic component consists essentially of M¹, M², M³, carbon, nitrogen, and optionally sulfur.

B8. The process of any of B1 to B7, wherein r1is a number in the range from 0.9 to 1.1.

B9. The process of any of B1 to B8, wherein at least one of the following is met:

(i) the feed has a H2 to CO molar ratio ranging from 0.3 to 3.0, preferably 0.5 to 2.5;

(ii) in step (I), the conversion conditions comprises at least one of the following:

a temperature in a range from 175 to 350° C., preferably 180 to 300° C.;

a pressure in a range from 100 to 3,000 kilopascal absolute, preferably 100 to 2,000 kilopascal absolute; and

a GHSV ranging from 500 to 10,000 hour⁻¹, preferably 1,000 to 5,000 hour⁻¹.

B9. The process of any of B1 to B8, wherein the process further comprises, before step (I):

(I0) activating the catalyst composition in the presence of a H2-containing atmosphere or an inert atmosphere.

B10. The process of B9, wherein step (10) is performed in the presence of a H2-containing atmosphere selected from syngas, hydrogen, a mixture of H2 and an inert gas, and mixtures thereof.

B11. The process of B9 or B10, wherein step (10) is performed at a temperature in a range from 150 to 500° C.

B12. The process of B9, wherein step (10) is performed in the presence of an inert atmosphere selected N₂, He, Ne, Ar, Kr, and mixtures thereof.

B13. The process of B12, wherein step (10) is performed at a temperature in a range from 200 to 600° C.

B14. The process of any of B1 to B13, wherein in step (I), the conversion product mixture comprises linear alpha-olefins, in aggregate, from 40 to 95 mol %, based on the total moles of the reaction product mixture.

B15. The process of any of B1 to B14, wherein in step (I), at least one of the following is met:

(i) the conversion product mixture comprises 1-butene from 40 to 95 mol %, based on the total moles of the C4 compounds in the conversion product mixture;

(ii) the conversion product mixture comprises 1-pentene from 40 to 95 mol %, based on the total moles of the C5 compounds in the conversion product mixture;

(iii) the conversion product mixture comprises 1-hexene from 40 to 95 mol %, based on the total moles of the C6 compounds in the conversion product mixture;

(iv) the conversion product mixture comprises 1-heptene from 40 to 95 mol %, based on the total moles of the C6 compounds in the conversion product mixture; and

(v) the conversion product mixture comprises 1-octene from 40 to 95 mol %, based on the total moles of the C8 compounds in the conversion product mixture.

B16. The process of any of B1 to A15, wherein in step (I), at least one of the following is met:

(i) the conversion product mixture comprises n-butane from 5 to 50 mol %, based on the total moles of the C4 compounds in the conversion product mixture;

(ii) the conversion product mixture comprises n-pentane from 5 to 50 mol %, based on the total moles of the C5 compounds in the conversion product mixture;

(iii) the conversion product mixture comprises n-hexane from 5 to 50 mol %, based on the total moles of the C6 compounds in the conversion product mixture;

(iv) the conversion product mixture comprises n-heptane from 5 to 50 mol %, based on the total moles of the C7 compounds in the conversion product mixture;

and

(v) the conversion product mixture comprises n-octane from 5 to 50 mol %, based on the total moles of the C8 compounds in the conversion product mixture.

B17. The process of any of B1 to B16, wherein in step (I), at least one of the following is met:

(i) the conversion product mixture comprises 2-butene from 0 to 20 mol %, based on the total moles of the C4 compounds in the conversion product mixture;

(ii) the conversion product mixture comprises C5 internal olefins from 0 to 20 mol %, based on the total moles of the C5 compounds in the conversion product mixture;

(iii) the conversion product mixture comprises C6 internal olefins from 0 to 20 mol %, based on the total moles of the C6 compounds in the conversion product mixture;

(iv) the conversion product mixture comprises C7 internal olefins from 0 to 20 mol %, based on the total moles of the C7 compounds in the conversion product mixture;

and

(v) the conversion product mixture comprises C8 internal olefins from 0 to 20 mol %, based on the total moles of the C8 compounds in the conversion product mixture.

1. A process for making a linear alpha-olefin product, the process comprising:
(I) contacting a feed comprising syngas with a catalyst composition in a conversion reactor under conversion conditions to produce a conversion product mixture comprising a linear alpha-olefin, wherein the catalyst composition comprises:
a support; and
a plurality of nanoparticles on the support, wherein:
each nanoparticle comprises a kernel, the kernels have an average particle size from 4 to 100 nm and a particle size distribution of no greater than 20%; the kernels comprise oxygen, a metal element M1, optionally sulfur, optionally phosphorus, an optional metal element M2, and optionally a third metal element M3, where:
M1 is selected from Mn, Fe, Co, and combination of two or more thereof,
M2 is selected from Ni, Zn, Cu, Mo, W, Ag, and combinations thereof;
M3 is selected from Y, Sc, alkaline metals, the lanthanides, group 13, 14, or 15 elements, and combinations thereof; and
the molar ratios of M2, M3, S, and P, if any, to M1 is r1, r2, r3, and r4, respectively, 0≤r1≤2, 0≤r2≤2, 0≤r3≤5, and 0≤r4≤5; and
(II) obtaining the linear alpha-olefin product from the conversion product mixture.

2. The process of claim 1, wherein 0.05≤r1≤0.5, and 0.005≤r2≤0.5.

3. The process of claim 1, wherein the kernels comprise an oxide of the at least one metal element selected from M1, M2, and M3.

4. The process of claim 1, wherein the nanoparticles have an average particle size of from 4 to 20 nm.

5. The process of claim 1, wherein the nanoparticles have a particle size distribution of from 5 to 15%.

6. The process of claim 1, wherein the kernels comprise at least two metal elements.

7. The process of claim 1, wherein the nanoparticles are substantially free of long-chain groups attached to the surface of the kernels.

8. The process of claim 1, wherein the kernels consist essentially of oxygen, M1, optionally M2, optionally M3, optionally sulfur, and optionally phosphorus.

9. The process of claim 1, wherein the kernels are substantially spherical.

10. The process of claim 1, wherein the kernels are rod-shaped.

11. The process of claim 1, wherein at least one of the following is met:
(i) the feed has a H2 to CO molar ratio ranging from 0.3 to 3.0;
(ii) in step (I), the conversion conditions comprises at least one of the following:
a temperature in a range from 175 to 350° C.;
a pressure in a range from 100 to 3,000 kilopascal absolute; and
a GHSV ranging from 500 to 10,000 hour⁻¹.

12. The process of claim 1, wherein the process further comprises, before step (I):
(I0) activating the catalyst composition in the presence of a H2-containing atmosphere.

13. The process of claim 1, wherein in step (I), the conversion product mixture comprises linear alpha-olefins, in aggregate, from 40 to 95 mol %, based on the total moles of the reaction product mixture.

14. The process of claim 1, wherein in step (I), at least one of the following is met:
(i) the conversion product mixture comprises 1-butene from 40 to 95 mol %, based on the total moles of the C4 compounds in the conversion product mixture;
(ii) the conversion product mixture comprises 1-pentene from 40 to 95 mol %, based on the total moles of the C5 compounds in the conversion product mixture;
(iii) the conversion product mixture comprises 1-hexene from 40 to 95 mol %, based on the total moles of the C6 compounds in the conversion product mixture;
(iv) the conversion product mixture comprises 1-heptene from 40 to 95 mol %, based on the total moles of the C7 compounds in the conversion product mixture;
(v) the conversion product mixture comprises 1-octene from 40 to 95 mol %, based on the total moles of the C8 compounds in the conversion product mixture;
(vi) the conversion product mixture comprises n-butane from 5 to 50 mol %, based on the total moles of the C4 compounds in the conversion product mixture;
(vii) the conversion product mixture comprises n-pentane from 5 to 50 mol %, based on the total moles of the C5 compounds in the conversion product mixture;
(viii) the conversion product mixture comprises n-hexane from 5 to 50 mol %, based on the total moles of the C6 compounds in the conversion product mixture;
(ix) the conversion product mixture comprises n-heptane from 5 to 50 mol %, based on the total moles of the C7 compounds in the conversion product mixture;
(x) the conversion product mixture comprises n-octane from 5 to 50 mol %, based on the total moles of the C8 compounds in the conversion product mixture;
(xi) the conversion product mixture comprises 2-butene from 0 to 20 mol %, based on the total moles of the C4 compounds in the conversion product mixture;
(xii) the conversion product mixture comprises C5 internal olefins from 0 to 20 mol %, based on the total moles of the C5 compounds in the conversion product mixture;
(xiii) the conversion product mixture comprises C6 internal olefins from 0 to 20 mol %, based on the total moles of the C6 compounds in the conversion product mixture;
(xiv) the conversion product mixture comprises C7 internal olefins from 0 to 20 mol %, based on the total moles of the C7 compounds in the conversion product mixture; and
(xv) the conversion product mixture comprises C8 internal olefins from 0 to 20 mol %, based on the total moles of the C8 compounds in the conversion product mixture.

15. The process of claim 1, wherein the conversion produce mixture is substantially free of linear 1-alcohols.

16. The process of claim 1, wherein the catalyst composition is made by a catalyst fabrication process comprising:
(A) providing a nanoparticle dispersion comprising a liquid medium and a plurality of nanoparticles distributed therein, wherein each nanoparticle comprises a kernel, the kernels have an average particle size from 4 to 100 nm and a particle size distribution of 20% or less; the kernels comprise oxygen, a metal element M1, optionally sulfur, optionally phosphorus, optionally a second metal element M2, and optionally a third metal element M3, where:
M1 is selected from Mn, Fe, Co, or a combination of two or more thereof;
M2 is selected from Ni, Zn, Cu, Mo, W, Ag, and combinations thereof;
M3 is selected from Y, Sc, alkaline metals, the lanthanides, group 13, 14, and 15 elements, and combinations thereof; and
the molar ratios of M2, M3, S, and P, if any, to M1 is r1, r2, r3, and r4, respectively, 0≤r1≤2, 0≤r2≤2, 0≤r3≤5, and 0≤r4≤5; and
(B) disposing a quantity of the nanoparticle dispersion on a support to obtain a supported nanoparticle composition.

17. The process of claim 16, wherein the catalyst fabrication process further comprises:
(C) calcining the supported nanoparticle composition to obtain a catalyst composition.

18. The process of claim 16, wherein the catalyst fabrication process further comprises:
(D) impregnating the support, the supported nanoparticle composition, or catalyst composition with a precursor of a promoter to obtain a catalyst precursor; and
(E) drying and/or calcining the catalyst precursor to obtain a catalyst composition comprising a promoter.

19. The process of claim 16, wherein step (A) comprises:
(A1) providing a first dispersion system at a first temperature, the first dispersion system comprising a salt of a long-chain organic acid and M1, optionally a salt of the long-chain organic acid and M2 optionally a salt of the long-chain organic acid and M3, a long-chain hydrocarbon solvent, optionally a salt of a second organic acid and M1,optionally a salt of a third organic acid and M2, optionally a salt of a fourth organic acid and M3, optionally sulfur or an organic sulfur compound soluble in the long-chain hydrocarbon solvent, and optionally an organic phosphorous compound soluble in the long-chain hydrocarbon solvent; and
(A2) heating the first dispersion system to a second temperature higher than the first temperature but no higher than the boiling point of the long-chain hydrocarbon solvent, where at least a portion of the salt(s) of the long-chain organic acid and at least a portion of the salt(s) of the second organic acid, if present, to form a second dispersion system comprising nanoparticles dispersed in the long-chain hydrocarbon solvent, and the nanoparticles comprise kernels, and the kernels comprise M1, optionally M2, optionally M3, oxygen, optionally sulfur, and optionally phosphorus;
(A3) separating the nanoparticles from the second dispersion system; and
(A4) dispersing the nanoparticles separated in (A3) in the liquid medium to form the nanoparticle dispersion.

20. The process of, wherein the nanoparticles have an average particle size in a range from 4 to 20 nm, and a particle size distribution of no greater than 20%.

21. The process of claim 19, wherein steps (A1), (A2), (A3), and (A4) are performed in the same vessel.

22. The process of claim 16, wherein the second organic acid has a boiling point lower than the first temperature.

23. The process of claim 16, wherein the first dispersion system is substantially free of a surfactant other than the salt(s) of the long-chain organic acid.

24. The process of claim 16, wherein the second temperature is in a range from 210° C. to 450° C.

25. The process of claim 16, wherein the long-chain organic acid and the long-chain hydrocarbon solvent do not differ in number of average carbon atoms per molecule by more than 4.
